SEQUENCE LISTING (17 sequences)

SEQ ID NO: 1 - protein, 107 aa, Homo sapiens
DIQMTQSPSSLSASVGDRVTITCRASQSISNNLHWYQQKPGKAPKLLIKYVFQSISGVPSRFSGSGSGTDFTFTISSLQPEDFATYYCQQSNSWPLTFGGGTKVEIK

SEQ ID NO: 2 - protein, 120 aa, Homo sapiens
QMQLVQSGPEVKKPGTSVKVSCKASGFTFTDYSMNWVRQARGQRLEWIGFIRNKANDYTTEYSASVKGRVTITRDMSTSTAYMELSSLRSEDTAVYYCARYPRYHAMDSWGQGTSVTVSS

SEQ ID NO: 3 - protein, 107 aa, Homo sapiens
DIQMTQSPSSLSASVGDRVTITCRASQSISNNLHWYQQKPGKAPKLLIKYAFQSISGVPSRFSGSGSGTDFTFTISSLQPEDFATYYCQQANSWPLTFGGGTKVEIK

SEQ ID NO: 4 - protein, 120 aa, Homo sapiens
QMQLVQSGPEVKKPGTSVKVSCKASGFTFDDYSMNWVRQARGQRLEWIGFIRNKANDYTTEYSASVKGRVTITRDMSTSTAYMELSSLRSEDTAVYYCARYPRHHAMDSWGQGTSVTVSS

SEQ ID NO: 5 - protein, 112 aa, Homo sapiens
DIQMTQSPSSLSASVGDRVTITCKSSQSLLYSNGKTYLNWYQQKPGKAPKLLIYLVSKLDSGVPSRFSGSGSGTDFTFTISSLQPEDIATYYCVQGSHFPWTFGQGTKVEIK

SEQ ID NO: 6 - protein, 115 aa, Homo sapiens
EVQLLESGGGLVQPGGSLRLSCAASGFTFSGYYMHWVRQAPGKGLEWVSYISCYNGVTSYNQKFKGRFTISRDNSKNTLYLQMNSLRAEDTAVYYCAKSHAMDYWGQGTLVTVSS

SEQ ID NO: 7 - DNA, 46 bp, Artificial
  oligonucleotide used for construction of vector
agggggtctagagggggtcaaaagaccccctctagaccccctttta

SEQ ID NO: 8 - DNA, 46 bp, Artificial
  oligonucleotide used for construction of vector
gattgctctagagtgcgacaaaagtcgcactctagagcaatcatta

SEQ ID NO: 9 - DNA, 33 bp, Artificial
  oligonucleotide used for construction of vector
cactacccgtacgacgttccggactacgcttct

SEQ ID NO: 10 - protein, 11 aa, Artificial
  polypeptide encoded by oligonucleotide used for construction of vector
HYPYDVPDYAS

SEQ ID NO: 11 - DNA, 24 bp, Artificial
  oligonucleotide used for construction of vector
gattacaaggatgacgacgataag

SEQ ID NO: 12 - protein, 8 aa, Artificial
  polypeptide encoded by oligonucleotide used for construction of vector
DYKDDDDK

SEQ ID NO: 13 - protein, 18 aa, Artificial
  polypeptide encoded by oligonucleotide used for construction of vector
MKKLLFAIPLVVPFYSHS

SEQ ID NO: 14 - DNA, 63 bp, Artificial
  oligonucleotide used for construction of vector
ggcgttacccaagccaaggagacagtcataatgcaaatgcagctggtgcagtctgggcctgag

SEQ ID NO: 15 - DNA, 63 bp, Artificial
  oligonucleotide used for construction of vector
ctcaggcccagactgcaccagctgcatttgcattatgactgtctccttggcttgggtaacgcc

SEQ ID NO: 16 - DNA, 66 bp, Artificial
  oligonucleotide used for construction of vector
gattacgccaagcttgcatgcggagaaaataaaatggacatccagatgacccagtctccatcctcc

SEQ ID NO: 17 - DNA, 66 bp, Artificial
  oligonucleotide used for construction of vector
ggaggatggagactgggtcatctggatgtccattttattttctccgcatgcaagcttggcgtaatc

(19) United States
(12) Patent Application Publication
(10) Pub. No.: US 2005/0181479 A1
(43) Pub. Date: Aug. 18, 2005

(54) Periplasmic expression of antibodies using a single signal sequence

(75) Inventors: William Dall'Acqua, Gaithersburg, MD (US); Herren Wu, Boyds, MD (US); Melissa Damschroder, Germantown, MD (US)

Correspondence Address: JOHNATHAN KLEIN-EVANS, ONE MEDIMMUNE WAY, GAITHERSBURG, MD, 20878, US

(73) Assignee: MEDIMMUNE, INC., Gaithersburg, MD (US)

(21) Appl. No.: 11/071,304

(22) Filed: Mar. 4, 2005

(51) Int. Cl. C07H 21/04; C07K 16/18; C12N 5/06; C12N 15/09

(52) U.S. Cl. 435/69.1; 435/320.1; 435/326; 530/388.1; 536/23.53

(57) ABSTRACT

The present invention relates to recombinant polynucleotides, expression vectors and methods for the production of multimeric proteins. The vectors and methods are useful for the production of multimeric protein and are unique in that they utilize a minimal number of signal sequences. More specifically, the present invention provides recombinant polynucleotide molecules and expression vectors comprising a promoter region operably linked to a transcription unit. The transcription unit is characterized by at least two DNA sequences encoding distinct polypeptides wherein at least one but not all DNA sequences further encodes a signal sequence operably linked to the DNA sequence encoding a polypeptide. The invention further provides methods of producing a multimeric protein using the expression vectors of the present invention.

### CROSS REFERENCE TO RELATED APPLICATIONS

This application claims the benefit under 35 U.S.C. §119(e) of United States Provisional Application ______ Attorney Docket Number AE704P1, filed Feb. 28, 2005, the disclosure of which is incorporated by reference in its entirety for all purposes.

### FIELD OF THE INVENTION

Expression in the bacterial periplasm is a very convenient route to express foreign recombinant proteins. The present invention relates to methods for the expression of multimeric proteins, which are polypeptide complexes consisting of at least two separate molecules, such as antibodies and antibody fragments (e.g., Fv and Fab) in bacteria using a single signal sequence.

### BACKGROUND OF THE INVENTION

Antibodies have a high degree of specificity and a broad target range, characteristics which make them useful tools in basic research, clinical and industrial use, where they serve as tools to selectively recognize virtually any kind of substrate. Numerous techniques to generate antibodies and/or antibody fragments have been developed (for overview see, Antibodies: A Laboratory Manual, Cold Spring Harbor Laboratory Press, NY; Harlow et al., 1989). One commonly used recombinant approach is the generation and/or “maturation” of antibody fragments by screening phage display antibody libraries derived from immunoglobulin sequences. Techniques and protocols required to generate, propagate, screen (pan), and use the antibody fragments from such libraries have been compiled (See, e.g., Barbas et al., 2001, Phage Display: A Laboratory Manual, Cold Spring Harbor Laboratory Press and Kay et al. (eds.), 1996, Phage Display of Peptides and Proteins: A Laboratory Manual, Academic Press, Inc., also see, Winter et al. U.S. Pat. No. 6,225,447 and Knappik et al. U.S. Pat. No. 6,300,064; Kufer et al. PCT publication WO 98/46645; Barbas et al. U.S. Pat. No. 6,096,551; and Kang et al. U.S. Pat. No. 6,468,738 each of which is incorporated herein by reference in its entirety.) Typically, once a useful phage clone is isolated from a phage library the next step is to express the antibody fragment (e.g., Fab and Fv fragments) on a small scale in a bacterial system (e.g., in Escherichia coli) for confirmation of its antigen binding specificity and/or characterization of its binding properties. Those clones possessing the desired properties can then be used to generate full length antibodies by cloning the variable or complementarity determining regions from the displaying phage into an antibody expression vector containing the antibody constant and/or framework regions to generate a complete antibody and then expressing the full length antibody in a prokaryotic or a eukaryotic host cell.

The typical antibody and most functional fragments thereof (e.g., IgG, Fab, Fv) are multimeric proteins composed of two or more distinct subunits, which in the case of antibodies and many other multimeric proteins are translated as separate polypeptides and then assembled. Current accepted methodologies for recombinant expression of multimeric proteins follows the two genes for two polypeptides rule. Thus, the transcription, translation and cellular localization or secretion of each polypeptide is controlled independently of the other polypeptide(s). As such, each polypeptide chain of a multimeric protein is controlled by separate promoters and for secreted proteins each polypeptides must contain a secretory leader sequence. However, this can lead to an imbalance in the ratio of the two polypeptide chains being expressed. In the case of antibodies in particular, this can lead to the production of aberrant molecules such as light chain dimers. Expression vectors incorporating a single promoter and a dicistronic messages have been developed and are commonly used in an effort to balance the production of multiple polypeptide chains by linking the transcription of the subunits. However, balanced production is rarely achieved exclusively by the use of such vectors. Thus it is often necessary to either manipulate the expression and/or growth conditions in order to optimize the production of the properly assembled multimeric protein or purify the assembled multimeric protein away from any free (e.g., unassembled) subunits. Optimization and purification are time consuming steps that can involve the construction of numerous expression vectors, laborious manipulation of culture conditions and multiple manipulations of samples.

In the case of secreted proteins (e.g., antibodies) the situation is complicated even further as each subunit of the multimeric protein must be produced and transported out of the cell. For the recombinant production of secreted proteins it has been well accepted that each polypeptides must contain its own secretory leader sequence, also referred to as a signal sequence or leader sequence, for efficient production of secreted product (see for example, Raffi, 2002, Methods Mol. Bio. 178:343-8). Signal sequences are relatively short (16-40 amino acids) in most species. The presence of a signal sequence on the protein permits the transport of the protein into the periplasm (prokaryotic hosts) or the secretion of the protein (eukaryotic hosts); generally little or no polypeptide is secreted in the absence of such a signal. One strategy that has been utilized for the production of recombinant secreted polypeptides is to express the polypeptides without signal sequences. The resulting material is then produced in the cytoplasm and often accumulates as insoluble “inclusion bodies” (Williams et al., Science 215:687-688, 1982; Schoner et al., Biotechnology 3:151-154, 1985), which can be readily purified. However, polypeptides accumulated in the form of inclusion bodies are relatively useless for screening purposes in biological or biochemical assays, or as pharmaceutical agents. Conversion of this insoluble material into active, soluble polypeptide requires slow and difficult solubilization and refolding protocols which often greatly reduce the net yield of biologically active polypeptide. These methods, which are generally not efficient for the production of monomeric polypeptides, are even less so for multimeric proteins and are rarely utilized for their production. Thus, signal sequences are incorporated into each subunit of a secreted multimeric protein to be produced and the problems associated with balancing production of each subunit remain a stumbling block to production.

The optimization of codon usage is another method that has been utilized specifically for the balanced expression of subunits (specifically antibody heavy and light chains) in a prokaryote system (Humphreys et al., 2002, Protein Expression and Purif. 26:309-20). This method however, requires the generation of small plasmid libraries of codon usage variants, which must be screened, and as such it is not useful for the rapid production of multimeric proteins.

While extensive optimization of polypeptide expression may be needed when production yields are desired, for screening purposes large quantities of a multimeric protein are often unnecessary. For screening purposes it is generally sufficient to eliminate the production of those aberrant multimers and/or free subunits which can interfere with the screening procedure (e.g., antibody light chain dimers). However, the elimination of aberrant multimers and/or free subunits often requires optimization of polypeptide expression and/or the addition of purification steps prior to screening. The present invention provides a novel strategy for the production of recombinant multimeric proteins consisting of at least two different subunits (i.e., Fv and Fab antibody fragments) which minimizes the production of aberrant multimers. Thus, the present invention can facilitate the rapid screening of large numbers of potential multimeric proteins (e.g., Fabs) by reducing or eliminated the need for laborious and time consuming optimization and/or purification prior to screening.

Citation or discussion of a reference herein shall not be construed as an admission that such is prior art to the present invention.

### SUMMARY OF THE INVENTION

The present invention provides recombinant polynucleotides, expression vectors and methods for the production of multimeric proteins (e.g., antibodies and fragments thereof). The vectors and methods are useful for the production of multimeric protein and are unique in that they utilize a minimal number of signal sequences. The vectors and methods of the present invention are particularly useful for the small scale production of recombinant antibody fragments in a prokaryotic host.

The present invention provides recombinant polynucleotide molecules comprising a promoter region operably linked to a transcription unit. The transcription unit is characterized by at least two DNA sequences encoding polypeptides wherein at least one but not all DNA sequences further encodes a signal sequence operably linked to the DNA sequence encoding a polypeptide. In one embodiment, the transcription unit is characterized by at least two DNA sequences encoding distinct polypeptides. In a preferred embodiment, the DNA sequences encode immunoglobulin polypeptides (e.g., light and heavy chains or fragments thereof) that can assemble to form antibodies or fragments thereof which are capable of binding an antigen.

The present invention further provides recombinant expression vectors comprising the isolated or recombinant polynucleotide molecules of the invention. In a preferred embodiment, the expression vectors allow for the expression of antibodies or fragments thereof. In a more preferred embodiment, the expression vectors of the present invention are useful for the production of secreted antibodies or fragments thereof.

The present invention also provides methods of producing a multimeric protein comprising culturing a host cell that has been transformed with a recombinant expression vector of the invention under conditions such that said host cell producing said multimeric protein. In another embodiment, the produced multimeric protein may be recovered from one or more of the following locations, including but not limited to, the periplasm, the whole cell and the culture media in which the host cell was cultured. In a preferred embodiment, said host cell secretes said multimeric protein. In another preferred embodiment, the method is used for the production of antibodies or fragments thereof.

The present invention additionally provides methods for reducing the production of free immunoglobulin light chain (i.e., light chain not in association with heavy chain) or a fragment thereof, during the expression of antibodies or fragments thereof.

Additional methods provided by the present invention include methods for reducing the accumulation of free immunoglobulin heavy chain (i.e., heavy chain not in association with light chain) or a fragment thereof during the production of antibodies or fragments thereof and methods for increasing the ration of active antibody or fragment thereof to total immunoglobulin chains or fragments thereof during the production of antibodies or fragments thereof.

### BRIEF DESCRIPTION OF THE FIGURES

FIG. 1 is an alignment of the amino acid sequences of the light (V_{L}) chains and variable heavy (V_{H}) of three anti-human EphA2 antibodies. The antibodies are designated G5, 12G3 and clone #9 (light chain SEQ ID NOS.: 1, 3 and 5, heavy chain SEQ ID NOS.: 2, 4 and 6, respectively). The boxed regions indicated the CDRs as defined by Kabat.

FIG. 2 depicts the details of the cloning region of the two-leader sequence phage vector used for expression of Fab fragments. A) is a schematic of the vector showing a promoter (Lac O/P), a first leader sequence (g3), a first cloning site (Palindromic loop 1), a light chain constant region (Cκ), a first tag(s) sequence (FLAG), and a stop codon followed by a second leader sequence, a second cloning site (Palindromic loop 2), a heavy chain constant region (C_{H}1), a second tag(s) sequence (HA and His₆) and a stop codon. The V_{L} and V_{H} genes are cloned in frame with the first constant domain of a human kappa (κ) light chain and the constant domain a human gamma1 (γ1) heavy chain, respectively. B) Lists the DNA sequences of the two cloning sites, Palindromic loops 1 and 2 (SEQ ID NOS.: 7 and 8, respectively), the DNA and amino acid sequences of the HA and FLAG tags (SEQ ID NOS.: 9, 10, 11 and 12, respectively) and the amino acid sequences of the g3 leader sequence (SEQ ID NOS.: 13).

FIG. 3 depicts thee variants of the two-leader sequence phage vector described in FIG. 2. A) The vector as described in FIG. 2 showing the variable light (V_{L}) and variable heavy (V_{C}) chain regions cloned into the first and second cloning regions, respectively (designated WT). B) A one-leader sequence variant (designated ΔL) with the first leader sequence removed such that the light chain will be produced without a leader sequence. C) A one-leader sequence variant (designated ΔH) with the second leader sequence removed such that the heavy chain will be produced without a leader sequence. D) A variant with no leader sequences (designated ΔLΔH) with both the first and second leader sequences removed such that neither immunoglobulin chain will be produced with a leader sequence.

FIG. 4 is a graph of the results of an EphA2-specific capture ELISA assay (described in Example 1) was used to determine if functional anti-EphA2 Fab fragments were being produced from each of the leader sequence variant expression vectors. The supernatant, as well as periplasmic and whole cell extracts where examined. The data indicate that similar levels of functional anti-EphA2 Fab were captured from samples in which the Fab was expressed from the WT and ΔL vectors. Little or no functional anti-EphA2 Fab was captured from samples in which the Fab was expressed from either the ΔH or ΔLΔH vectors.

### DETAILED DESCRIPTION OF THE INVENTION

The present invention is based, in part, on the unexpected discovery that functional multimeric proteins, defined herein as polypeptide complexes composed of two or more distinct polypeptides, can be produced and secreted when only one of the distinct polypeptides (also referred to herein as “subunit(s)”) is operatively linked to a signal sequence. Additionally, the present invention demonstrates that by manipulating which subunit is linked to a signal sequence the ratios of the different subunits produced can be modulated. The inventors have further determined that the methods and vectors of the invention can be used to reduce the undesirable accumulation of free subunits (i.e., a subunit that is not assembled into the multimeric protein) thereby minimizing a common source of contamination and/or toxic accumulation during the production of multimeric proteins. Thus, the methods and vectors provided facilitate the production of multimeric proteins without the need for extensive optimization methods to balance the production of each subunit and/or without requiring sample purification to remove excess free subunits. Additionally, the methods and vectors provided can overcome certain difficulties encountered when the production of free subunits is toxic to the host cell.

Accordingly, the present invention relates to vectors and methods for the production of multimeric proteins (e.g., antibodies or fragments thereof). The vectors and methods of the present invention are particularly useful for the small scale production of recombinant antibody fragments.

Polynucleotide Molecules and Expression Vectors

The present invention provides recombinant polynucleotide molecules useful for the production of multimeric proteins. In one embodiment, the recombinant polynucleotide molecules of the invention utilize a polycistronic expression system characterized by the use of a promoter region operably linked to a transcription unit which encodes multiple distinct polypeptides (i.e., subunits) which together make up the multimeric protein. In a specific embodiment, the recombinant polynucleotide molecules of the invention utilize a dicistronic expression system characterized by the use of a promoter region operably linked to a transcription unit which encodes two distinct subunits.

In one embodiment, the present invention provides recombinant polynucleotide molecules comprising a promoter region operably linked to a transcription unit, wherein the transcription unit is characterized by at least two DNA sequences encoding distinct subunits, wherein at least one but not all the DNA sequences further encode a signal sequence operably linked to the DNA sequence encoding a subunit. In a preferred embodiment, the recombinant polynucleotide molecules of the invention comprise a promoter region operably linked to a transcription unit, said transcription unit comprising a first DNA sequence encoding a first subunit and a second DNA sequence encoding a second subunit, wherein, either the first DNA sequence or the second DNA sequence but not both, additionally encode a secretion signal operably linked to the DNA sequence encoding said first or second subunit. In a specific embodiment, said first DNA sequence, but not said second DNA sequence, additionally encodes a secretion signal operably linked to the DNA sequence encoding said first subunit. In another specific embodiment, said second DNA sequence, but not said first DNA sequence, additionally encodes a secretion signal operably linked to the DNA sequence encoding said second subunit.

In a preferred embodiment, said first DNA sequence encodes an immunoglobulin light chain or a fragment thereof and said second DNA sequence encodes an immunoglobulin heavy chain or a fragment thereof. In an another preferred embodiment, said first DNA sequence encodes an immunoglobulin heavy chain or a fragment thereof and said second DNA sequence encodes an immunoglobulin light chain or a fragment thereof. In a most preferred embodiment, the immunoglobulin heavy chain or a fragment thereof and the immunoglobulin light chain or a fragment thereof encoded by the recombinant polynucleotide molecules of the present invention can assemble into a multimeric protein which is capable of binding an antigen.

In one embodiment, the transcription unit comprises at least two, or at least three, or at least four, or at least five, DNA sequences encoding distinct subunits wherein at least one but not all the DNA sequences further encode a signal sequence operably linked to the DNA sequence encoding a subunit. In one embodiment, the transcription unit comprises two DNA sequences encoding distinct subunits wherein only one of the DNA sequences further encodes a signal sequence operably linked to the DNA sequence encoding a subunit. In another embodiment, the transcription unit comprises three DNA sequences encoding distinct subunits wherein one or two of the DNA sequences further encodes a signal sequence operably linked to the DNA sequences encoding distinct subunits. In still another embodiment, the transcription unit comprises four DNA sequences encoding distinct subunits wherein one, or two, or three of the DNA sequences further encodes a signal sequence operably linked to the DNA sequences encoding distinct subunits. In yet another embodiment, the transcription unit comprises five DNA sequences encoding distinct subunits wherein one, or two, or three, or four of the DNA sequences further encodes a signal sequence operably linked to the DNA sequences encoding distinct subunits.

In yet another embodiment, the recombinant polynucleotide molecules of the invention utilize multiple promoters for the production of multimeric proteins. Without wishing to be bound by any particular theory, the use of multiple promoters may be preferable for the expression of multimeric proteins in eukaryotic systems and in some prokaryotic systems (see for example, Raffi, 2002, Methods Mol. Bio. 178:343-8). Situations where the use of multiple promoters for the production of multimeric proteins would be preferable are known to one skilled in the art. A number of possible configurations are possible including but not limited to, a separate promoter operably linked to each DNA sequence encoding a each distinct subunit of the multimeric protein, a separate promoter operably linked to individual transcription units each of which encodes at least two distinct subunits and a combination of promoters operably linked to individual DNA sequences and promoters operably linked to individual transcription units.

In one embodiment, the isolated or recombinant polynucleotide molecules of the invention comprise more then one promoter region, wherein each promoter region is separately operably linked to a DNA sequence encoding a distinct subunit. In one embodiment, a recombinant polynucleotide molecule of the invention comprises a first promoter operably linked to a first DNA sequence encoding a first subunit and a second promoter operably linked to a second DNA sequence encoding a second subunit, wherein either said first DNA sequence or said second DNA sequence but not both, additionally encode a secretion signal operably linked to the DNA sequence encoding said first or second subunit. It is contemplated that a recombinant polynucleotide of the invention may comprise more then two promoters operably linked to individual DNA sequences. In a specific embodiment, said first subunit, encoded by said first DNA sequence, is an immunoglobulin light chain or a fragment thereof and said second subunit, encoded by said second DNA sequence, is an immunoglobulin heavy chain or a fragment thereof. In an another specific embodiment, said first subunit, encoded by said first DNA sequence, is an immunoglobulin heavy chain or a fragment thereof and said second subunit, encoded by said second DNA sequence, is an immunoglobulin light chain or a fragment thereof.

It is also contemplated that the recombinant polynucleotide molecules of the invention may comprise multiple promoters operably linked to multiple transcription units, wherein at least one promoter is operably linked to each transcription unit and wherein each transcription unit is characterized by at least two DNA sequences encoding distinct subunits and wherein at least one but not all the DNA sequences of all the transcription units further encodes a signal sequence operably linked to the DNA sequence encoding a subunit. It is further contemplated that the isolated or recombinant polynucleotide molecules of the invention may comprise a mixture of: i) promoters operably linked to transcription units and ii) promoters operably linked to individual DNA sequences encoding distinct subunits of the multimeric protein, wherein at least one but not all the DNA sequences of the recombinant polynucleotide molecule further encodes a signal sequence operably linked to the DNA sequence encoding a subunit.

It is known in the art that signal sequences may be more or less effective in their ability to direct a protein for secretion. It is contemplated that a weak or poor signal sequence may be used in place of no signal sequence in all of the above embodiments. The relative efficacy of signal sequence can be readily determined by one skilled in the art.

In still another embodiment, one or more or all of the DNA sequences encoding a distinct subunit can be fused to one or more polynucleotide sequence encoding a peptide (i.e., peptide tag or epitope tag) to facilitate purification of the subunit produced. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., 1989, PNAS 86:821, for instance, hexa-histidine provides for convenient purification of the fusion protein. Other peptide tags useful for purification include, but are not limited to, the hemagglutinin “HA” tag, which corresponds to an epitope derived from the influenza hemagglutinin protein (Wilson et al., 1984, Cell 37:767) and the “flag” tag. Methods for incorporating peptide tags are well known in the art see, for example, Chapter 10 in Current Protocols in Molecular Biology, F. M. Ausubel et al., ed., John Wiley & Sons (Chichester, England, 1998) and Example 1 infra.

In a preferred embodiment, the recombinant polynucleotide molecules of the invention are incorporated into an expression vector (referred to herein as “expression vector(s) of the invention”). Expression vectors generally contain elements necessary to maintain the expression vector within a host (e.g., origin of replication or autonomously replicating sequence) and for selection of host cells that contain the vector (e.g., selectable marker). In addition, an expression vector may also provide elements necessary for the transcription and translation of the multimeric proteins encoded by the recombinant polynucleotide molecules of the invention. A variety of host-vector systems may be utilized in the present invention and are well known to one skilled in the art. These include but are not limited to mammalian cell systems infected with virus (e.g., vaccinia virus, adenovirus, etc.); insect cell systems infected with virus (e.g., baculovirus); microorganisms such as yeast (e.g., Saccharomyces Pichia) containing yeast vectors; or bacteria (e.g., E. coli and B. subtilis) transformed with bacteriophage, DNA, plasmid DNA, or cosmid DNA (see for example, Chapters 1, 13 and 16 in Current Protocols in Molecular Biology, F. M. Ausubel et al., ed., John Wiley & Sons (Chichester, England, 1998)). The expression elements of vectors vary in their strengths and specificities. Depending on the host-vector system utilized, any one of a number of suitable transcription and translation elements may be used.

In one embodiment, one or more recombinant polynucleotide molecules of the invention are incorporated into a single vector. In another embodiment, recombinant polynucleotide molecules of the invention are incorporated into more then one vector. For example, to produce a multimeric protein consisting of three separate subunits, DNA encoding each subunit is operably linked to a separate promoter and each promoter-DNA unit is incorporated into the same or separate expression vectors. Other possible variations include, but are not limited to, a first promoter operably linked to a transcription unit encoding a first subunit and a second subunit may be incorporated into one expression vector and a second promoter operably linked to a DNA encoding a third subunit may be incorporated into a separate expression vector. It is specifically contemplated, for any combination of one or more expression vectors utilized for the expression of a multimeric polypeptide, that at least one but not all the DNA sequences encoding a subunit further encode a signal sequence operably linked to the DNA sequence encoding the subunit. When more then one expression vector is utilized they may contain identical or different elements for maintenance and/or selection although they should be compatible for the same host cell.

In one embodiment, a single expression vector of the invention comprises a recombinant polynucleotide molecule of the invention comprising a first promoter operably linked to a first DNA sequence encoding a first subunit and a second promoter operably linked to a second DNA sequence encoding a second subunit, wherein either said first DNA sequence or said second DNA sequence but not both, additionally encode a secretion signal operably linked to the DNA sequence encoding said first or second subunit. In a specific embodiment, said first subunit, encoded by said first DNA sequence, is an immunoglobulin light chain or a fragment thereof and said second subunit, encoded by said second DNA sequence, is an immunoglobulin heavy chain or a fragment thereof. In a separate embodiment, said first subunit, encoded by said first DNA sequence, is an immunoglobulin heavy chain or a fragment thereof and said second subunit, encoded by said second DNA sequence, is an immunoglobulin light chain or a fragment thereof.

In another embodiment, a first expression vector of the invention comprises a first recombinant polynucleotide of the invention comprising a first promoter operably linked to a first DNA sequence encoding a first subunit and a second expression vector of the invention comprises a second recombinant polynucleotide of the invention comprising a second promoter operably linked to a second DNA sequence encoding a second subunit, wherein either said first DNA sequence or said second DNA sequence but not both, additionally encode a secretion signal operably linked to the DNA sequence encoding said first or second subunit. In a specific embodiment, said first subunit, encoded by said first DNA sequence, is an immunoglobulin light chain or a fragment thereof and said second subunit, encoded by said second DNA sequence, is an immunoglobulin heavy chain or a fragment thereof. In a separate embodiment, said first subunit, encoded by said first DNA sequence, is an immunoglobulin heavy chain or a fragment thereof and said second subunit, encoded by said second DNA sequence, is an immunoglobulin light chain or a fragment thereof.

In yet another embodiment, a single expression vector of the invention comprises a recombinant polynucleotide molecule of the invention comprising a promoter region operably linked to a transcription unit, said transcription unit comprising a first DNA sequence encoding a first subunit and a second DNA sequence encoding a second subunit, wherein, either the first DNA sequence or the second DNA sequence but not both, additionally encode a secretion signal operably linked to the DNA sequence encoding said first or second subunit. In a specific embodiment, said first subunit, encoded by said first DNA sequence, is an immunoglobulin light chain or a fragment thereof and said second subunit, encoded by said second DNA sequence, is an immunoglobulin heavy chain or a fragment thereof. In a separate embodiment, said first subunit, encoded by said first DNA sequence, is an immunoglobulin heavy chain or a fragment thereof and said second subunit, encoded by said second DNA sequence, is an immunoglobulin light chain or a fragment thereof. In a preferred embodiment, the expression vector of the invention is an M13-based phage vector which allows the expression of antibody Fab fragments that contain the first constant domain of the human γ1 heavy chain and the constant domain of the human light chain under the control of the lacZ promoter (Wu & An, 2003, Methods Mol. Biol., 207, 213-233; Wu, 2003, Methods Mol. Biol., 207, 197-212; both of which are incorporated herein by reference in their entireties and detailed in Example 1, supra).

Expression vectors containing the recombinant polynucleotide molecules of the invention can be identified by three general approaches: (a) nucleic acid hybridization, (b) presence or absence of “marker” gene functions, and (c) expression of inserted sequences. In the first approach, the presence of a gene encoding a peptide, polypeptide, protein or a fusion protein in an expression vector can be detected by nucleic acid hybridization using probes comprising sequences that are homologous to an inserted gene encoding the peptide, polypeptide, protein or the fusion protein, respectively. In the second approach, the recombinant vector/host system can be identified and selected based upon the presence or absence of certain “marker” gene functions (e.g., thymidine kinase activity, resistance to antibiotics, transformation phenotype, occlusion body formation in baculovirus, etc.) caused by the insertion of a recombinant polynucleotide molecule of the invention in the vector. For example, if the recombinant polynucleotide molecule of the invention is inserted within the marker gene sequence of the vector, recombinants containing the insert can be identified by the absence of the marker gene function. In the third approach, recombinant expression vectors can be identified by assaying for the production of the multimeric protein (e.g., antibody or fusion protein) expressed by the recombinant vector. Such assays can be based, for example, on the physical or functional properties of the multimeric protein in in vitro assay systems, e.g., binding with an antibody that recognizes the multimeric protein.

Expression vectors of the invention may be introduced into a host cells (a process defined herein as, “Transformation”) by various methods which are well known in the art. The method is selected based on the type of host cell being transformed and may include, but is not limited to, viral infection, electroporation, heat shock, lipofection, and particle bombardment. Such “transformed” cells include stably transformed cells in which the inserted DNA is capable of replication either as an autonomously replicating plasmid or as part of the host chromosome. They also include cells which transiently express the inserted DNA or RNA for limited periods of time. A host cell may be co-transfected with one more expression vectors of the invention.

Signal Sequences and Promoters

The signal sequence provided in the recombinant polynucleotide molecules and expression vectors of the invention is a polypeptide present at the N-terminus of a polypeptide useful in aiding in the secretion of the polypeptide to the outside of the host. Also called “leading peptide,” or “leader sequence.” Without wishing to be bound by any particular theory, the presence of a signal sequence on the protein facilitates the transport of the protein into the periplasm (prokaryotic hosts) or the secretion of the protein (eukaryotic hosts). In both prokaryotes and eukaryotes, the signal sequence is generally removed from the amino-terminus of the protein molecule by enzymatic cleavage during transport of the polypeptide through the membrane. In prokaryotes, the signal sequence directs the nascent protein across the inner membrane into the periplasmic space which may also allow proper folding of some proteins that cannot fold properly in the cytoplasm. Transport to the periplasmic space also functions as a partial purification step, as the periplasm contains fewer proteins than does the cytoplasm. Proteins present in the periplasm may be released by a mild osmotic shock of the bacterial cells. E. coli cells which express the kil gene product may be used to achieve the secretion of proteins transported to the periplasm without the need for cell lysis or osmotic shock [Kobayashi, T. et al., J. Bacteriol. 166:728 (1986)]. Signal sequences from bacterial or eukaryotic genes are highly conserved in terms of function, although not in terms of sequence, although many of these sequences have been shown to be interchangeable (Grey et al., 1985, Gene 39:247).

Numerous signal sequences which may be incorporated into the isolated or recombinant polynucleotide molecules of the invention are well known in the art (see for example, Pugsley, 1993, Microbiol. Rev., 57:50-108, 1993; Simonen et al., 1993, Microbiol. Rev., 57:109-137; Pines et al., 1999, Mol Biotechnol 12:25-34; Nothwehr et al., 1990, Bioessays 12:479-84; Oka et al., 1985, Proc. Natl. Acad. Sci. USA. 82: 7212; PCT publication WO 03/068956 and U.S. Pat. Nos. 4,336,336; 508,4384; 5,576,195 each of which is incorporated herein by reference in its entirety). Generally, the choice of signal sequence is determined in part by the choice of host cell. Bacterial signal sequences include, but are not limited to, bacteria phage gene 3 protein (g3), pectate lyase (pel), phosphatase (pho), maltose-binding protein (malE), major outer membrane proteins (lamB, ompF, ompA and ompC) and alkaline phosphatase (alkP). Eukaryotic signal sequences include but are not limited to, eukaryotic viral signal sequences (e.g., gp70 from MMLV), yeast signal sequences (e.g., Carboxypeptidase Y, KRE5 protein, Glycolipid anchored surface protein precursor) and mammalian signal sequences (e.g., Immunoglobulin chain, Ceruloplasmin precursor, Chromogranin precursor, beta-hexosaminidase a-chain precursor).

The expression of a transcription unit and/or a DNA sequence can be placed under control of any of a large number of promoter regulatory sequences known to one skilled in the art. Promoters which may be used include, but are not limited to, the SV40 early promoter region (Bemoist and Chambon, 1981, Nature 290:304-310), the promoter contained in the 3′ long terminal repeat of Rous sarcoma virus (Yamamoto, et al., 1980, Cell 22:787-797), the herpes thymidine kinase promoter (Wagner et al., 1981, Proc. Natl. Acad. Sci. USA 78:1441-1445), the regulatory sequences of the metallothionein gene (Brinster et al., 1982, Nature 296:39-42), the tetracycline (Tet) promoter (Gossen et al., 1995, Proc. Nat. Acad. Sci. USA 89:5547-5551); prokaryotic expression vectors such as the β-lactamase promoter (Villa-Kamaroff et al., 1978, Proc. Natl. Acad. Sci. USA 75:3727-3731), or the tac promoter (DeBoer et al., 1983, Proc. Natl. Acad. Sci. USA 80:21-25; see also “Useful proteins from recombinant bacteria” in Scientific American, 1980, 242:74-94); plant expression vectors comprising the nopaline synthetase promoter region (Herrera-Estrella et al., Nature 303:209-213) or the cauliflower mosaic virus ³⁵S RNA promoter (Gardner et al., 1981, Nucl. Acids Res. 9:2871), and the promoter of the photosynthetic enzyme ribulose biphosphate carboxylase (Herrera-Estrella et al., 1984, Nature 310:115-120); promoter elements from yeast or other fingi such as the Gal 4 promoter, the ADC (alcohol dehydrogenase) promoter, PGK (phosphoglycerol kinase) promoter, alkaline phosphatase promoter, and the following animal transcriptional control regions, which exhibit tissue specificity and have been utilized in transgenic animals: elastase I gene control region which is active in pancreatic acinar cells (Swift et al., 1984, Cell 38:639-646; Omitz et al., 1986, Cold Spring Harbor Symp. Quant. Biol. 50:399-409; MacDonald, 1987, Hepatology 7:425-515); insulin gene control region which is active in pancreatic beta cells (Hanahan, 1985, Nature 315:115-122), immunoglobulin gene control region which is active in lymphoid cells (Grosschedl et al., 1984, Cell 38:647-658; Adames et al., 1985, Nature 318:533-538; Alexander et al., 1987, Mol. Cell. Biol. 7:1436-1444), mouse mammary tumor virus control region which is active in testicular, breast, lymphoid and mast cells (Leder et al., 1986, Cell 45:485-495), albumin gene control region which is active in liver (Pinkert et al., 1987, Genes and Devel. 1:268-276), alpha-fetoprotein gene control region which is active in liver (Krumlauf et al., 1985, Mol. Cell. Biol. 5:1639-1648; Hammer et al., 1987, Science 235:53-58; alpha 1-antitrypsin gene control region which is active in the liver (Kelsey et al., 1987, Genes and Devel. 1: 161-171), beta-globin gene control region which is active in myeloid cells (Mogram et al., 1985, Nature 315:338-340; Kollias et al., 1986, Cell 46:89-94; myelin basic protein gene control region which is active in oligodendrocyte cells in the brain (Readhead et al., 1987, Cell 48:703-712); myosin light chain-2 gene control region which is active in skeletal muscle (Sani, 1985, Nature 314:283-286); neuronal-specific enolase (NSE) which is active in neuronal cells (Morelli et al., 1999, Gen. Virol. 80:571-83); brain-derived neurotrophic factor (BDNF) gene control region which is active in neuronal cells (Tabuchi et al., 1998, Biochem. Biophysic. Res. Com. 253:818-823); glial fibrillary acidic protein (GFAP) promoter which is active in astrocytes (Gomes et al., 1999, Braz J Med Biol Res 32(5): 619-631; Morelli et al., 1999, Gen. Virol. 80:571-83) and gonadotropic releasing hormone gene control region which is active in the hypothalamus (Mason et al., 1986, Science 234:1372-1378).

Producing Multimeric Proteins

The present invention also provides methods of producing a multimeric protein by culturing a host cell that has been transformed with at least one expression vector of the in invention under conditions such that said host cell produces said multimeric protein. In a preferred embodiment, said host cell secretes said multimeric protein. Multimeric proteins which may be produced by the methods of the invention include, but are not limited to, antibodies or fragments thereof. In one preferred embodiment, the method is used for the production of antibodies or fragments thereof. In another preferred embodiment, the multimeric protein is recovered. The produced multimeric protein may be recovered from one or more of the following locations, including but not limited to, the periplasm, the whole cell and the culture media in which the host cell was cultured. In a preferred embodiment, the multimeric protein is recovered from the periplasm and/or the culture media in which the host cell was cultured.

In one embodiment, at least a portion of the multimeric protein produced utilizing the vectors and methods of the present invention will be properly assembled and have at least one expected functional activity. The term “functional activity”, when used in reference to a multimeric protein, refers to a biological, biochemical and/or cellular activity that the multimeric protein performs. Functional activity encompasses activities that the multimeric protein performs in its native cellular location as well as activities it performs in an artificial setting (e.g., in vitro or ex vivo). Such activities include, but are not limited to, enzymatic activity (e.g., kinase or phosphatase activity), binding activity (e.g., antigen, ligand or receptor binding), biological activity (e.g., ability to elicit a particular biological response when delivered to a cell or subject such as inhibition or stimulation of cell growth) and combinations thereof. In a preferred embodiment, at least 1%, or at least 5%, or at least 10%, or at least 20%, or at least 30%, or at least 40%, or at least 50%, or at least 60%, or at least 70%, or at least 80%, or at least 90%, or at least 100% of the multimeric protein produced utilizing the vectors and methods of the present invention will be properly assembled and have at least one expected functional activity.

The present invention provides methods for reducing the production of free subunits (i.e., subunits not in association with the multimeric protein) during production of a multimeric protein. It is contemplated that reducing the production of free subunits will reduce or eliminate a number of complications known to arise due to the production/presence of free subunits including, but not limited to, toxic accumulation of free subunits, production of aberrant subunit aggregates (e.g., immunoglobulin light chain dimers), contamination of properly formed multimeric protein with free subunits or aberrant subunit aggregates. It is contemplated that one or more of the expression vectors of the present invention may be utilized to reduce the expression of free subunits during production of a multimeric protein. The choice of expression vector is determined in part by the host system being utilized and can be readily determined by one skilled in the art.

In one embodiment, the method of reducing the production of free subunits comprises culturing a host cell that has been transformed with at least one expression vector of the invention, wherein the DNA encoding at least one subunit whose production is to be reduced is not operably linked to a DNA encoding a signal sequence and wherein the DNA encoding at least one subunit whose production is not t be reduced is operably linked to a DNA encoding a signal sequence. In one embodiment, the production of free subunit produced during the production of a multimeric protein is reduced by at least 2 fold, or by at least 5 fold, or by at least 10 fold, or by at least 15 fold, or by at least 25 fold, or by at least 50 fold, or by at least 100 fold when compared to the amount of free subunit produced when said subunit is operably linked to a signal sequence.

The present invention additionally provides methods for reducing the production of free immunoglobulin light chain or a fragment thereof (i.e., light chain or fragment thereof not in association with heavy chain), during the production of antibodies or fragments thereof. In one embodiment, the method of reducing the production of free immunoglobulin light chain or a fragment thereof comprises culturing a host cell that has been transformed with at least one expression vector of the invention, wherein the DNA encoding a immunoglobulin light chain or fragment thereof is not operably linked to a DNA encoding a signal sequence and wherein the DNA encoding a immunoglobulin heavy chain or fragment thereof is operably linked to a DNA encoding a signal sequence. In a specific embodiment, the method of reducing the production of free immunoglobulin light chain or a fragment thereof comprises culturing a host cell that has been transformed with an expression vector of the invention, said expression vector comprising a promoter region operably linked to a transcription unit, said transcription unit comprising a DNA sequence encoding an immunoglobulin light chain or fragment thereof and a DNA sequence encoding a secretion signal operably linked to a DNA sequence encoding an immunoglobulin heavy chain or fragment thereof. In a preferred embodiment, the production of free immunoglobulin light chain or fragment thereof produced during the production of an antibody or fragment thereof is reduced by at least 2 fold, or by at least 5 fold, or by at least 10 fold, or by at least 15 fold, or by at least 25 fold, or by at least 50 fold, or by at least 100 fold when compared to the amount of free immunoglobulin light chain or fragment thereof produced when said subunit is operably linked to a signal sequence.

Additional methods provided by the present invention include methods for reducing the accumulation of free immunoglobulin heavy chain (i.e., heavy chain not in association with light chain) or a fragment thereof during the production of antibodies or fragments thereof. In one embodiment, the method of reducing the reducing the accumulation of free immunoglobulin heavy chain or a fragment thereof comprises culturing a host cell that has been transformed with at least one expression vector of the invention, wherein the DNA encoding a immunoglobulin heavy chain or fragment thereof is not operably linked to a DNA encoding a signal sequence and wherein the DNA encoding a immunoglobulin light chain or fragment thereof is operably linked to a DNA encoding a signal sequence. In a specific embodiment, the method of reducing the accumulation of free immunoglobulin heavy chain or a fragment thereof comprises culturing a host cell that has been transformed with an expression vector of the invention, said expression vector comprising a promoter region operably linked to a transcription unit, said transcription unit comprising a DNA sequence encoding an immunoglobulin heavy chain or fragment thereof and a DNA sequence encoding a secretion signal operably linked to a DNA sequence encoding an immunoglobulin light chain or fragment thereof. In a preferred embodiment, the production of free immunoglobulin heavy chain or fragment thereof produced during the production of an antibody or fragment thereof is reduced by at least 2 fold, or by at least 5 fold, or by at least 10 fold, or by at least 15 fold, or by at least 25 fold, or by at least 50 fold, or by at least 100 fold when compared to the amount of free immunoglobulin heavy chain or fragment thereof produced when said subunit is operably linked to a signal sequence.

Methods for increasing the ratio of functional antibody or functional fragment thereof to total immunoglobulin chains or fragments thereof during the production of antibodies or fragments thereof. The term “functional”, when used in reference to an antibody or fragment thereof, refers to a biological, biochemical and/or cellular activity that the antibody or fragment thereof performs. Without wishing to be bound by any particular theory, antibodies or fragments thereof which have assembled properly are generally have the most desirably functional activity. Functional activity encompasses activities that the multimeric protein performs in its native cellular location as well as activities it performs in an artificial setting (e.g., in vitro or ex vivo). Such activities include, but are not limited to binding activity (e.g., antigen binding), biological activity (e.g., effector functions such as those mediated by FcγR binding) and combinations thereof. Numerous biological assays for assaying antibody function are known in the art and several are detailed below in the section entitled “Biological Assays.”

In one embodiment, the method for increasing the ratio of functional antibody or functional fragment thereof to total immunoglobulin chains or fragments thereof comprises culturing a host cell that has been transformed with at least one expression vector of the invention, wherein the DNA encoding a immunoglobulin light chain or fragment thereof is not operably linked to a DNA encoding a signal sequence and wherein the DNA encoding a immunoglobulin heavy chain or fragment thereof is operably linked to a DNA encoding a signal sequence. In a specific embodiment, the method for increasing the ratio of functional antibody or fragment thereof to total immunoglobulin chains or fragments thereof comprises culturing a host cell that has been transformed with an expression vector of the invention, said expression vector comprising a promoter region operably linked to a transcription unit, said transcription unit comprising a DNA sequence encoding a secretion signal operably linked to a DNA sequence encoding an immunoglobulin light chain or fragment thereof and a DNA sequence encoding an immunoglobulin heavy chain or fragment thereof.

Once a multimeric protein has been produced it may be purified by any method known in the art for purification. For example an immunoglobulin molecule may be purified by known methods including, but not limited to, chromatography (e.g., ion exchange, affinity, particularly by affinity for the specific antigen after Protein A, and sizing column chromatography), centrifugation, differential solubility.

Host Cells

Host cells which can be used for the expression of multimeric polypeptides using the expression vectors and methods of the present invention are well know in the art and include, but are not limited to, mammalian cells, insect cells, plant cells, yeast, and bacteria. Appropriate cell lines or host systems can be chosen to ensure the desired modification and processing of the foreign protein expressed. For example, expression in a bacterial system will produce an unglycosylated product and expression in yeast will produce a glycosylated product. Eukaryotic host cells that possess the cellular machinery for proper processing of the primary transcript (e.g., acetylation, methylation, glycosylation, and phosphorylation) of the gene product may be used.

In one embodiment, the methods of the invention utilize bacterial host cells. Among bacterial hosts which may be utilized E. coli is a commonly used both for small scale screening as well as for large scale production of recombinant proteins. A number of particularly useful E. coli strains are commercially available including, for example, XL1-Blue (Stratagene®), JM101 and DH5a (New England BioLabs®). Other microbial strains which may be used include, but are not limited to, Bacillus subtilis, Salmonella typhimurium or Serratia marcescens, Kluyveromyces lactis, and various Pseudomonas species may be used. Methods for culturing bacterial hosts for the production of polypeptides are well known in the art, see for example, Current Protocols in Molecular Biology, F. M. Ausubel et al., ed., John Wiley & Sons (Chichester, England, 1998) at 16.1 to 16.8 and Protein Expression Technologies: Current Status and Future Trends, F. Baneyx, ed., Horizon Bioscience (Norwich, England, 2004) at chapters 2, 4 and 10.

Yeast is another preferred host, a number of different yeast host cells are know in the art including, but not limited to, Schizosaccharomyces pombe, Saccharomyces cerevisiae, and Saccharomyces Pichia. Yeast provides substantial advantages for the production of immunoglobulin light and heavy chains. Yeasts carry out post-translational peptide modifications including glycosylation. A number of recombinant DNA strategies now exist which utilize strong promoter sequences and high copy number plasmids which can be used for overt production of the desired proteins in yeast. Yeast recognizes leader sequences on cloned mammalian gene products and secretes peptides bearing leader sequences (i.e. prepeptides) (Hitzman, et al., 11th International Conference on Yeast, Genetics and Molecular Biology, Montpelier, France, Sep. 13-17, 1982).

Numerous mammalian host cells which may be utilized are known in the art including, but are not limited to, CHO, VERY, BHK, Hela, COS, MDCK, 293, 3T3, W138, NSO, and in particular, neuronal cell lines such as, for example, SK-N-AS, SK-N-FI, SK-N-DZ human neuroblastomas (Sugimoto et al., 1984, J. Natl. Cancer Inst. 73: 51-57), SK-N-SH human neuroblastoma (Biochim. Biophys. Acta, 1982, 704: 450-460), Daoy human cerebellar medulloblastoma (He et al., 1992, Cancer Res. 52: 1144-1148) DBTRG-05MG glioblastoma cells (Kruse et al., 1992, In Vitro Cell. Dev. Biol. 28A: 609-614), IMR-32 human neuroblastoma (Cancer Res., 1970, 30: 2110-2118), 1321N1 human astrocytoma (Proc. Natl. Acad. Sci. USA, 1977, 74: 4816), MOG-G-CCM human astrocytoma (Br. J. Cancer, 1984, 49: 269), U87MG human glioblastoma-astrocytoma (Acta Pathol. Microbiol. Scand., 1968, 74: 465-486), A172 human glioblastoma (Olopade et al., 1992, Cancer Res. 52: 2523-2529), C6 rat glioma cells (Benda et al., 1968, Science 161: 370-371), Neuro-2a mouse neuroblastoma (Proc. Natl. Acad. Sci. USA, 1970, 65: 129-136), NB41A3 mouse neuroblastoma (Proc. Natl. Acad. Sci. USA, 1962, 48: 1184-1190), SCP sheep choroid plexus (Bolin et al., 1994, J. Virol. Methods 48: 211-221), G355-5, PG-4 Cat normal astrocyte (Haapala et al., 1985, J. Virol. 53: 827-833), Mpf ferret brain (Trowbridge et al., 1982, In Vitro 18: 952-960), and normal cell lines such as, for example, CTX TNA2 rat normal cortex brain (Radany et al., 1992, Proc. Natl. Acad. Sci. USA 89: 6467-6471) such as, for example, CRL7030 and Hs578Bst.

The expression vectors of the invention are transferred to a host cell by conventional techniques and the transfected cells are then cultured by conventional techniques to produce a multimeric protein (e.g., antibody or fragment thereof). Thus, the invention includes host cells containing a recombinant polynucleotide and/or expression vector of the invention.

Multimeric Proteins

Multimeric proteins that can be encoded by the recombinant polynucleotides and expression vectors of the present invention include, but are not limited to, nearly any polypeptide complex composed of more then one distinct subunit. Without wishing to be bound by any particular theory, the intracellular environment does not facilitate the proper folding and/or assembly of protein which are normally secreted. Thus, the vectors and methods of the present invention are particularly useful for the production of secreted multimeric proteins and fragments thereof which cannot assume a functional conformation in the cytoplasm. It is also contemplated that the vectors and methods of the present invention may be used to produced polypeptides which are not necessarily found assembled into multimeric proteins but which are capable of assembling, for example upon a stimulatory signal and/or processing event (e.g., complement proteins). In one embodiment, the recombinant polynucleotides and expression vectors of the present invention encode immunoglobulin polypeptides (e.g., light and heavy chains or fragments thereof) that can assemble to form antibodies or fragments thereof which are capable of binding an antigen. In another embodiment, the expression vectors of the present invention allow for the production of antibodies or fragments thereof. In a preferred embodiment, the recombinant polynucleotides, expression vectors and methods of the present invention are useful for the production of secreted antibodies or fragments thereof.

Antibodies encoded by the recombinant polynucleotides and expression vectors of the present invention and produced by the method of the invention (infra) may include, but are not limited to, synthetic antibodies, monoclonal antibodies, recombinantly produced antibodies, intrabodies, multispecific antibodies, bispecific antibodies, human antibodies, humanized antibodies, chimeric antibodies, synthetic antibodies, Fab fragments, F(ab′) fragments, disulfide-linked Fvs (sdFv), anti-idiotypic (anti-Id) antibodies, and epitope-binding fragments of any of the above. In particular, antibodies encoded by the recombinant polynucleotides and expression vectors of the present invention and produced by the methods of the present invention include immunoglobulin molecules and immunologically active portions of immunoglobulin molecules. The immunoglobulin molecules can be of any type (e.g., IgG, IgE, IgM, IgD, IgA and IgY), class (e.g., IgG₁, IgG₂, IgG₃, IgG₄, IgA₁ and IgA₂) or subclass of immunoglobulin molecule.

Antibodies or antibody fragments encoded by the recombinant polynucleotides and expression vectors of the present invention and produced by the methods of the present invention may be from any animal origin including birds and mammals (e.g., human, murine, donkey, sheep, rabbit, goat, guinea pig, camel, horse, or chicken). Preferably, the antibodies are human or humanized monoclonal antibodies. As used herein, “human” antibodies include antibodies having the amino acid sequence of a human immunoglobulin and include antibodies isolated from human immunoglobulin libraries or from mice that express antibodies from human genes.

Antibodies or antibody fragments encoded by the recombinant polynucleotides and expression vectors of the present invention and produced by the methods of the present invention may be monospecific, bispecific, trispecific or of greater multispecificity. Multispecific antibodies may immunospecifically bind to different epitopes of desired target molecule or may immunospecifically bind to both the target molecule as well as a heterologous epitope, such as a heterologous polypeptide or solid support material. See, e.g., International Publication Nos. WO 93/17715, WO 92/08802, WO 91/00360, and WO 92/05793; Tutt, et al., 1991, J. Immunol. 147:60-69; U.S. Pat. Nos. 4,474,893, 4,714,681, 4,925,648, 5,573,920, and 5,601,819; and Kostelny et al., 1992, J. Immunol. 148:1547-1553 (which are incorporated herein by reference in their entireties).

Antibodies or fragments thereof encoded by the recombinant polynucleotides and expression vectors of the present invention and produced by the methods of the present invention encompasses single domain antibodies, including camelized single domain antibodies (see e.g., Muyldermans et al., 2001, Trends Biochem. Sci. 26:230; Nuttall et al., 2000, Cur. Pharm. Biotech. 1:253; Reichmann and Muyldermans, 1999, J. Immunol. Meth. 231:25; International Publication Nos. WO 94/04678 and WO 94/25591; U.S. Pat. No. 6,005,079; which are incorporated herein by reference in their entireties).

Antibodies or antibody fragments encoded by the recombinant polynucleotides and expression vectors of the present invention and produced by the methods of the present invention also encompass antibodies or fragments thereof that have half-lives (e.g., serum half-lives) in a mammal, preferably a human, of greater than 15 days, preferably greater than 20 days, greater than 25 days, greater than 30 days, greater than 35 days, greater than 40 days, greater than 45 days, greater than 2 months, greater than 3 months, greater than 4 months, or greater than 5 months. The increased half-lives of antibodies in a mammal, preferably a human, results in a higher serum titer of said antibodies or antibody fragments in the mammal, and thus, reduces the frequency of the administration of said antibodies or antibody fragments and/or reduces the concentration of said antibodies or antibody fragments to be administered. Antibodies or fragments thereof having increased in vivo half-lives can be generated by techniques known to those of skill in the art. For example, antibodies or fragments thereof with increased in vivo half-lives can be generated by modifying (e.g., substituting, deleting or adding) amino acid residues identified as involved in the interaction between the Fc domain and the FcRn receptor (see, e.g., International Publication No. WO 97/34631 and U.S. patent application Ser. No. 10/020,354, both of which are incorporated herein by reference in their entireties).

It is specifically contemplated that antibody-like and antibody-domain fusion proteins may also be produced using the recombinant polynucleotides, expression vectors and methods of the present invention. An antibody-like molecule is any molecule that has been generated with a desired binding property, see, e.g., International Publication No. WO 04/044011. Antibody-domain fusion proteins may incorporate one or more antibody domains such as the Fc domain or the variable domain. For example, the heterologous polypeptides may be fused or conjugated to a Fab fragment, Fd fragment, Fv fragment, F(ab)₂ fragment, a VH domain, a V_{L} domain, a VH CDR, a VL CDR, or fragment thereof. A large number of antibody-domain molecules are known in the art including, but not limited to, diabodies (dsFv)₂ (Bera et al., 1998, J. Mol. Biol. 281:475-83); minibodies (homodimers of scFv-CH3 fusion proteins), tetravalent di-diabody (Lu et al., 2003 J. Immunol. Methods 279:219-32), tetravalent bi-specific antibodies called Bs(scFv)4-IgG (Zuo et al., 2000, Protein Eng. 13:361-367). Fc domain fusions combine the Fc region of an immunoglobulin with a fusion partner which in general can be an protein, including, but not limited to, a ligand, an enzyme, the ligand portion of a receptor, an adhesion protein, or some other protein or domain. See, e.g., Chamow et al., 1996, Trends Biotechnol 14:52-60; Ashkenazi et al., 1997, Curr Opin Immunol 9:195-200; Heidaran et al., 1995, FASEB J. 9:140-5 (said references incorporated by reference in their entireties). Methods for fusing or conjugating polypeptides to antibody portions are well known in the art. See, e.g., U.S. Pat. Nos. 5,336,603, 5,622,929, 5,359,046, 5,349,053, 5,447,851, and 5,112,946; European Patent Nos. EP 307,434 and EP 367,166; International publication Nos. WO 96/04388 and WO 91/06570; Ashkenazi et al., 1991, Proc. Natl. Acad. Sci. USA 88: 10535-10539; Zheng et al., 1995, J. Immunol. 154:5590-5600; and Vil et al., 1992, Proc. Natl. Acad. Sci. USA 89:11337-11341 (said references incorporated by reference in their entireties).

Methods of Generating Antibodies

Antibodies or antibody fragments encoded by the recombinant polynucleotides and expression vectors of the present invention and produced by the methods of present invention can be generated by any method known in the art for the synthesis of antibodies, in particular, by chemical synthesis or preferably, by recombinant expression techniques.

Monoclonal antibodies which can be encoded by the recombinant polynucleotides and expression vectors of the present invention and produced by the methods of the present invention can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Antibodies: A Laboratory Manual, E. Harlow and D. Lane, ed., Cold Spring Harbor Laboratory Press (Cold Spring Harbor, N.Y., 1988); and Hammerling, et al., in: Monoclonal Antibodies and T-Cell Hybridomas 563-681 (Elsevier, N.Y., 1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art. Briefly, mice can be immunized with a antigen of interest, generally but not always a polypeptide such as a full length protein or a domain thereof (e.g., the extracellular domain) can be utilized, and once an immune response is detected, e.g., antibodies specific for the antigen of interest are detected in the mouse serum, the mouse spleen is harvested and splenocytes isolated. The splenocytes are then fused by well known techniques to any suitable myeloma cells, for example cells from cell line SP20 available from the ATCC. Hybridomas are selected and cloned by limited dilution. Additionally, a RIMMS (repetitive immunization, multiple sites) technique can be used to immunize an animal (Kilpatrick et al., 1997, Hybridoma 16:381-9, incorporated herein by reference in its entirety). Hybridoma clones are then assayed by methods known in the art for cells that secrete antibodies capable of binding a polypeptide of the invention. Ascites fluid, which generally contains high levels of antibodies, can be generated by immunizing mice with positive hybridoma clones.

Accordingly, monoclonal antibodies can be generated by culturing a hybridoma cell secreting an antibody of interest wherein, preferably, the hybridoma is generated by fusing splenocytes isolated from a mouse immunized with polypeptide of interest or fragment thereof with myeloma cells and then screening the hybridomas resulting from the fusion for hybridoma clones that secrete an antibody able to bind the polypeptide of interest.

A recombinant nucleotide or expression vector of the present invention encoding an antibody can be obtained from sequencing hybridoma clone DNA. If a clone containing a nucleic acid encoding a particular antibody or an epitope-binding fragment thereof is not available, but the sequence of the antibody molecule or epitope-binding fragment thereof is known, a nucleic acid encoding the immunoglobulin may be chemically synthesized or obtained from a suitable source (e.g., an antibody cDNA library, or a cDNA library generated from, or nucleic acid, preferably poly A+ RNA, isolated from any tissue or cells expressing the antibody, such as hybridoma cells selected to express an antibody) by PCR amplification using synthetic primers that hybridize to the 3′ and 5 ′ends of the sequence or by cloning using an oligonucleotide probe specific for the particular gene sequence to identify, e.g., a cDNA clone from a cDNA library that encodes the antibody. Amplified nucleic acids generated by PCR may then be cloned into replicable cloning vectors using any method well known in the art.

Once the nucleotide sequence of the antibody is determined, the nucleotide sequence of the antibody may be manipulated using methods well known in the art for the manipulation of nucleotide sequences, e.g. recombinant DNA techniques, site directed mutagenesis, PCR, etc. (see, for example, the techniques described in Current Protocols in Molecular Biology, F. M. Ausubel et al., ed., John Wiley & Sons (Chichester, England, 1998); Molecular Cloning: A Laboratory Manual, 3nd Edition, J. Sambrook et al., ed., Cold Spring Harbor Laboratory Press (Cold Spring Harbor, N.Y., 2001); Antibodies: A Laboratory Manual, E. Harlow and D. Lane, ed., Cold Spring Harbor Laboratory Press (Cold Spring Harbor, N.Y., 1988); and Using Antibodies: A Laboratory Manual, E. Harlow and D. Lane, ed., Cold Spring Harbor Laboratory (Cold Spring Harbor, N.Y., 1999) which are incorporated by reference herein in their entireties), to generate antibodies having a different amino acid sequence by, for example, introducing deletions, and/or insertions into desired regions of the antibodies.

Antibodies which can be encoding by the recombinant polynucleotides and expression vectors of the present invention can also be generated using various phage display methods known in the art. In phage display methods, functional antibody domains are displayed on the surface of phage particles that carry the polynucleotide sequences encoding them. In particular, DNA sequences encoding V_{H} and V_{L} domains are amplified from animal cDNA libraries (e.g., human or murine cDNA libraries of lymphoid tissues). The DNA encoding the V_{H} and V_{L} domains are recombined together with an scFv linker by PCR and cloned into a phagemid vector (e.g., p CANTAB 6 or pComb 3 HSS). The vector is electroporated in E. coli and the E. coli is infected with helper phage. Phage used in these methods are typically filamentous phage including fd and M13 and the V_{H} and V_{L} domains are usually recombinantly fused to either the phage gene III or gene VIII. Phage expressing an antigen binding domain that binds to the antigen epitope of interest can be selected or identified with antigen, e.g., using labeled antigen or antigen bound or captured to a solid surface or bead. Examples of phage display methods that can be used to generate antibodies which can be expressed using the recombinant polynucleotides, expression vectors and methods of the present invention include those disclosed in Brinkman et al., 1995, J. Immunol. Methods 182:41-50; Ames et al., 1995, J. Immunol. Methods 184:177; Kettleborough et al., 1994, Eur. J. Immunol. 24:952-958; Persic et al., 1997, Gene 187:9; Burton et al., 1994, Advances in Immunology 57:191-280; International Application No. PCT/GB91/01134; International Publication Nos. WO 90/02809, WO 91/10737, WO 92/01047, WO 92/18619, WO 93/11236, WO 95/15982, WO 95/20401, and WO97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743 and 5,969,108; each of which is incorporated herein by reference in its entirety.

After phage selection, the antibody coding regions from the phage are isolated and can be used to generate whole antibodies, including human antibodies as described in the references above and below. Antibody coding regions may also further manipulated by a number of techniques well known in the art for the maturation, optimization and/or humanization of antibodies or fragments thereof. Examples of methods which can be used for the maturation, optimization and/or humanization of antibodies or fragments thereof include those disclosed in Blaise et al., 2004, Gene 324:211-8; Fijii, 2004, Methods Mol Biol 248:345-59; Marks, 2004, Methods Mol Biol 248:327-43; Wu, 2003, Methods Enzymol. 197:212-; Wu et al., 2003, Methods Enzymol. 213:233 Wu et al., 1998, PNAS USA 95:6037-42; International Publication Nos. WO04/024871, WO04/070010, WO05/012877, WO03/088911 and U.S. Pat. No. 6,849,425; each of which is incorporated herein by reference in its entirety). It is contemplated that the recombinant polynucleotides, expression vectors and methods of the present invention are particularly useful for the screening of numerous antibodies or fragments thereof in conjunction with the maturation, optimization and/or humanization of one or more antibodies or fragments thereof.

It is specifically contemplated that for some uses, including in vivo use of antibodies in humans and in vitro detection assays, antibodies produced by the recombinant polynucleotides, expression vectors and methods of the present invention are preferably human or chimeric antibodies. Completely human antibodies are particularly desirable for therapeutic treatment of human subjects. Human antibodies can be made by a variety of methods known in the art including phage display methods described above using antibody libraries derived from human immunoglobulin sequences. See also U.S. Pat. Nos. 4,444,887 and 4,716,111; and International Publication Nos. WO 98/46645, WO 98/50433, WO 98/24893, WO 98/16654, WO 96/34096, WO 96/33735, and WO 91/10741; each of which is incorporated herein by reference in its entirety. Methods for producing chimeric antibodies are known in the art. See e.g., Morrison, 1985, Science 229:1202; Oi et al., 1986, BioTechniques 4:214; Gillies et al., 1989, J. Immunol. Methods 125:191-202; and U.S. Pat. Nos. 5,807,715, 4,816,567, and 4,816,397, CDR-grafting (EP 239,400; International Publication No. WO 91/09967; and U.S. Pat. Nos. 5,225,539, 5,530,101, and 5,585,089), veneering or resurfacing (EP 592,106; EP 519,596; Padlan, 1991, Molecular Immunology 28(4/5): 489-498; Studnicka et al., 1994, Protein Engineering 7:805; and Roguska et al., 1994, PNAS 91:969), and chain shuffling (U.S. Pat. No. 5,565,332). Each of the above references are incorporated herein by reference in their entirety.

Biological Assays

Multimeric proteins produced utilizing the vectors and methods of the present invention may be characterized in a variety of ways well-known to one of skill in the art. In particular, antibodies or fragments thereof produced utilizing the recombinant polynucleotides; expression vectors and methods of the present invention may be assayed for the ability to immunospecifically bind to an antigen. Such an assay may be performed in solution (e.g., Houghten, 1992, Bio/Techniques 13:412 421), on beads (Lam, 1991, Nature 354:82 84), on chips (Fodor, 1993, Nature 364:555 556), on bacteria (U.S. Pat. No. 5,223,409), on spores (U.S. Pat. Nos. 5,571,698; 5,403,484; and 5,223,409), on plasmids (Cull et al., 1992, Proc. Natl. Acad. Sci. USA 89:1865 1869) or on phage (Scott and Smith, 1990, Science 249:386 390; Cwirla et al., 1990, Proc. Natl. Acad. Sci. USA 87:6378 6382; and Felici, 1991, J. Mol. Biol. 222:301 310) (each of these references is incorporated herein in its entirety by reference).

Assays for immunospecific binding to a specific antigen and cross-reactivity with other antigens are well known in the art. Immunoassays which can be used to analyze immunospecific binding and cross-reactivity include, but are not limited to, competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well-known in the art (see, e.g., Current Protocols in Molecular Biology, F. M. Ausubel et al., ed., John Wiley & Sons (Chichester, England, 1998) which is incorporated by reference herein in its entirety). Exemplary immunoassays are described briefly below (but are not intended by way of limitation).

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the antibody of interest to the cell lysate, incubating for a period of time (e.g., 1-4 hours) at 4.degree. C., adding protein A and/or protein G sepharose beads to the cell lysate, incubating for about an hour or more at 4° C., washing the beads in lysis buffer and resuspending the beads in SDS/sample buffer. The ability of the antibody of interest to immunoprecipitate a particular antigen can be assessed by, e.g., western blot analysis. One of skill in the art would be knowledgeable as to the parameters that can be modified to increase the binding of the antibody to an antigen and decrease the background (e.g., pre-clearing the cell lysate with sepharose beads). For further discussion regarding immunoprecipitation protocols see, e.g., Current Protocols in Molecular Biology, F. M. Ausubel et al., ed., John Wiley & Sons (Chichester, England, 1998) at 10.16.1.

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), blocking the membrane with primary antibody (the antibody of interest) diluted in blocking buffer, washing the membrane in washing buffer, blocking the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., ³²P or ¹²⁵I) diluted in blocking buffer, washing the membrane in wash buffer, and detecting the presence of the antigen. One of skill in the art would be knowledgeable as to the parameters that can be modified to increase the signal detected and to reduce the background noise. For further discussion regarding western blot protocols see, e.g., Current Protocols in Molecular Biology, F. M. Ausubel et al., ed., John Wiley & Sons (Chichester, England, 1998) at 10.8.1.

ELISAs comprise preparing antigen, coating the well of a 96 well microtiter plate with the antigen, adding the antibody of interest conjugated to a detectable compound such as an enzymatic substrate (e.g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognizes the antibody of interest) conjugated to a detectable compound may be added to the well. Further, instead of coating the well with the antigen, the antibody may be coated to the well. In this case, a second antibody conjugated to a detectable compound may be added following the addition of the antigen of interest to the coated well. One of skill in the art would be knowledgeable as to the parameters that can be modified to increase the signal detected as well as other variations of ELISAs known in the art. For further discussion regarding ELISAs see, e.g., Current Protocols in Molecular Biology, F. M. Ausubel et al., ed., John Wiley & Sons (Chichester, England, 1998) 11.2.1.

The binding affinity of an antibody to an antigen and the off-rate of an antibody-antigen interaction can be determined by competitive binding assays. One example of a competitive binding assay is a radioimmunoassay comprising the incubation of labeled antigen with the antibody of interest in the presence of increasing amounts of unlabeled antigen, and the detection of the antibody bound to the labeled antigen. The affinity of the antibody of interest for a particular antigen and the binding off-rates can be determined from the data by scatchard plot analysis. Competition with a second antibody can also be determined using radioimmunoassays. In this case, the antigen is incubated with antibody of interest conjugated to a labeled compound in the presence of increasing amounts of an unlabeled second antibody.

Techniques to determine the ability of an antibody or fragment thereof to inhibit the binding of an antigen to its host cell receptor are well known to those of skill in the art. For example, cells expressing a receptor can be contacted with a ligand for that receptor in the presence or absence of an antibody or fragment thereof that is an antagonist of the ligand and the ability of the antibody or fragment thereof to inhibit the ligand's binding can measured by, for example, flow cytometry or a scintillation assay. The ligand or the antibody or antibody fragment can be labeled with a detectable compound such as a radioactive label (e.g., ³²P, ³⁵S, and ¹²⁵I) or a fluorescent label (e.g., fluorescein isothiocyanate, rhodamine, phycoerythrin, phycocyanin, allophycocyanin, o-phthaldehyde and fluorescamine) to enable detection of an interaction between the ligand and its receptor. Alternatively, the ability of antibodies or fragments thereof to inhibit a ligand from binding to its receptor can be determined in cell-free assays. For example, a ligand can be contacted with an antibody or fragment thereof that is an antagonist of the ligand and the ability of the antibody or antibody fragment to inhibit the ligand from binding to its receptor can be determined. Preferably, the antibody or the antibody fragment that is an antagonist of the ligand is immobilized on a solid support and the ligand is labeled with a detectable compound. Alternatively, the ligand is immobilized on a solid support and the antibody or fragment thereof is labeled with a detectable compound. A ligand may be partially or completely purified (e.g., partially or completely free of other polypeptides) or part of a cell lysate. Alternatively, a ligand can be biotinylated using techniques well known to those of skill in the art (e.g., biotinylation kit, Pierce Chemicals; Rockford, Ill.).

### EXAMPLES

The invention is now described with reference to the following examples. These examples are provided for the purpose of illustration only and the invention should in no way be construed as being limited to these examples but rather should be construed to encompass any and all variations which become evident as a result of the teachings provided herein.

### Example 1

Antibody Expression Using a Single Signal Sequence

It had been observed that during the expression of Ig fragments a major component of the resulting Ig fragments produced are in fact “free” light chain, that is to say light chain that was not found in association with a corresponding heavy chain (Humphreys et al., 2002, Protein Expression and Purif. 26:309-20 and Table 1). To investigate alternative methods of expressing Igs in a host cell a series of expression vector constructs were generated using only one or no signal sequences (e.g., a signal sequence only on the light or heavy chain). Our results indicate that the removal of either the heavy chain signal sequence or both signal sequences result in only minimal production of active Fab. In contrast however, removal of the light chain signal sequence results in a significant reduction in the amount of “free” light chain produced but had virtually no effect on the detection of active Fab as assayed by an antigen-specific capture ELISA. These data indicate that the use of only a heavy chain signal sequence when expressing Ig molecules, particularly in prokaryotic host cells, can facilitate the production of a more homogeneous population of properly assembled and active Ig molecules.

Materials and Methods

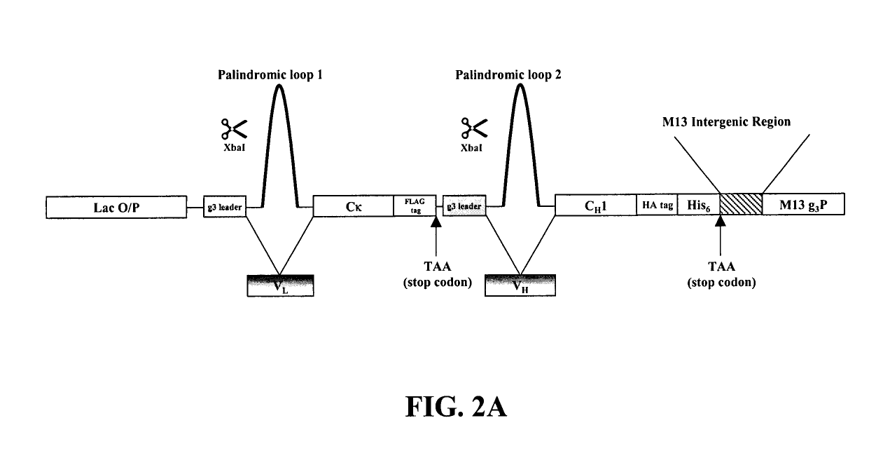

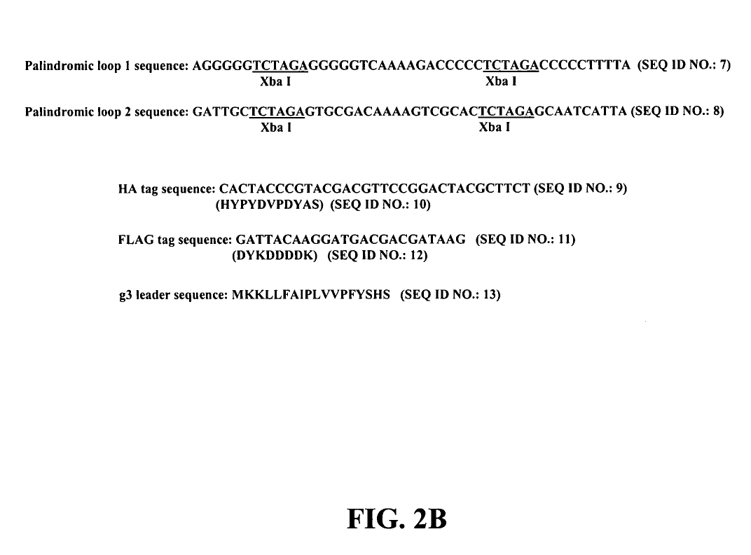

Cloning of Fab Fragments: Cloning of the different Fab fragments into the phage expression vector (FIG. 2) was carried out by hybridization mutagenesis (Kunkel et al., 1987, Methods Enzymol. 154:367-382) as described in Wu, 2003, Methods Enzymol. 197:212. Briefly, the V regions of G5, 12G3 and an irrelevant antibody were synthesized by PCR so that they contained sequences specific to the end of the vector's corresponding leader sequences and the beginning of the vector's corresponding constant regions. Minus single-stranded DNA corresponding to these various V regions was then purified by ethanol precipitation after dissociation of double-stranded PCR-synthesized product using sodium hydroxide and elimination of the biotinylated strand by streptavidin-coated magnetic beads as described (Wu et al., 2003, Methods Enzymol. 213:233 and Wu, ibid). These different strands were then individually annealed to the two palindromic loop regions of the phage expression vector (FIG. 2). Those loops contain a unique XbaI site which allows for the selection of the vectors that contain both V_{L} and V_{H} chains fused in frame with human kappa (κ) constant and first human γ1 constant regions, respectively (Wu et al. and Wu, both ibid). Synthesized DNA was then electroporated into XL1-Blue cells for plaque cloning and phage production as described (Wu, ibid).

Generation of the Leader Sequence Variants: Deletion (Δ) of the leader sequences in front of the heavy (ΔH) and/or light (ΔL) chains of G5 and 12G3 Fabs was carried out as described below. The following primers were used:

Primer # 1

(SEQ ID NO.: 14)

5′-GGCGTTACCCAAGCCAAGGAGACAGTCATAATGCAAATGCAGCTGGT
GCAGTCTGGGCCTGAG-3′
Primer # 2

(SEQ ID NO.: 15)

5′-CTCAGGCCCAGACTGCACCAGCTGCATTTGCATTATGACTGTCTCCT
TGGCTTGGGTAACGCC-3′
Primer # 3

(SEQ ID NO.: 16)

5′-GATTACGCCAAGCTTGCATGCGGAGAAAATAAAATGGACATCCAGAT
GACCCAGTCTCCATCCTCC-3′
Primer # 4

(SEQ ID NO.: 17)

5′-GGAGGATGGAGACTGGGTCATCTGGATGTCCATTTTATTTTCTCCGC
ATGCAAGCTTGGCGTAATC-3′

For ΔLG5, ΔLΔHG5 and ΔLΔH12G3 variants, deletions were introduced using the QuickChange XL site-directed mutagenesis Kit (Stratagene, La Jolla, Calif.) according to the manufacturer's instructions and the 3/4, 1/2/3/4 or 1/2/3/4 primer combinations, respectively. The appropriate Fab-encoding phage vector (G5 or 12G3, see “Cloning of the Fab Fragments,” supra) was used as the template. Synthesized DNA was then electroporated into XL1-Blue cells for plaque cloning and phage production as described (Wu, ibid).

For ΔHG5, ΔL12G3 and ΔH12G3 variants, deletions were introduced by hybridization mutagenesis (Kunkel et al., ibid) using primers 2, 4 and 2, respectively. The appropriate Fab-encoding phage vector (G5 or 12G3, see “Cloning of the Fab Fragments,” supra) was used as the template. Synthesized DNA was then electroporated into XL1-Blue cells for plaque cloning and phage production as described (Wu, ibid).

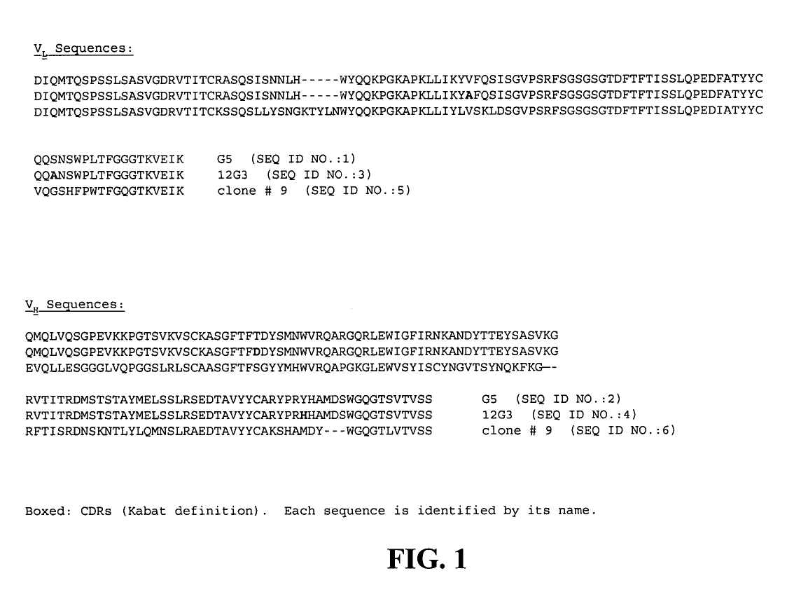

Expression and Purification of a V_{L}-C_{L} Standard: The light chain variable (V_{L}) and constant regions (C_{L}) of the anti-EphA2 monoclonal antibody clone # 9 (FIG. 1) were cloned into a mammalian expression vector encoding a human cytomegalovirus major immediate early (hCMVie) enhancer, promoter and 5′-untranslated region (Boshart et al., 1985, Cell 41:521-530). In this system, a full length human kappa (κ) chain is secreted (Johnson et al., 1997, J. Infect. Dis. 176:1215-1224). This construct was expressed transiently in human embryonic kidney (HEK) 293 cells and harvested 72 hours post-transfection. The secreted, soluble V_{L}-C_{L} was purified from the conditioned media directly on protein L (Pierce, Ill.) according to the manufacturer's instructions. The purified kappa light chain (typically >95% homogeneity, as judged by SDS-PAGE) was dialyzed against phosphate buffered saline (PBS), flash frozen and stored at −70° C. Protein concentration was calculated by the bicinchoninic acid method.

Production of G5 and 12G3 Fab Standards: G5 and 12G3 Fab standards were generated from the corresponding chimeric human IgG1 versions of G5 and 12G3 using an ImmunoPure Fab Preparation Kit (Pierce, Ill.) according to the manufacturer's instructions. The purified Fabs were dialyzed against phosphate buffered saline (PBS), flash frozen and stored at −70° C. Protein concentrations were calculated by the bicinchoninic acid method.

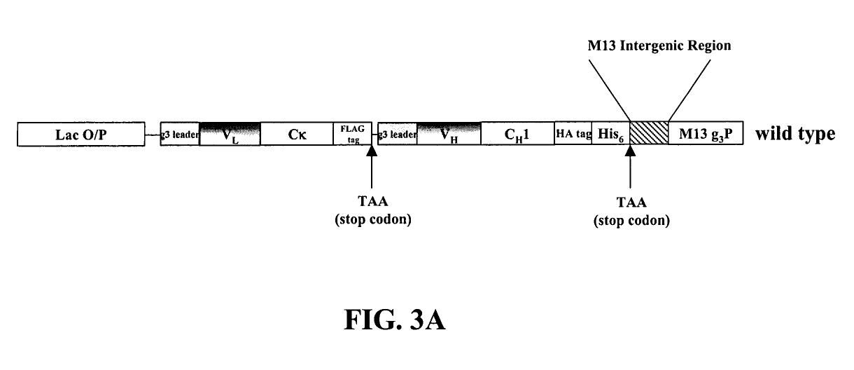

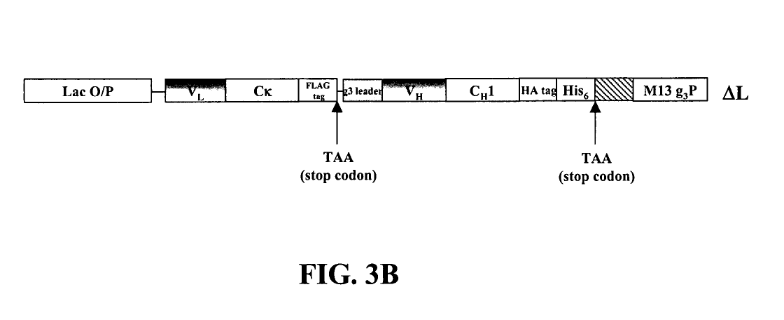

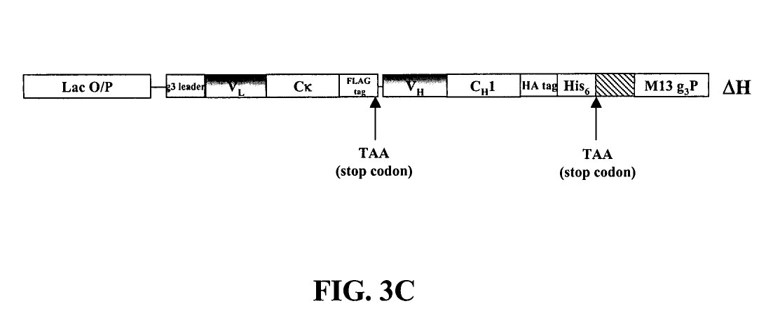

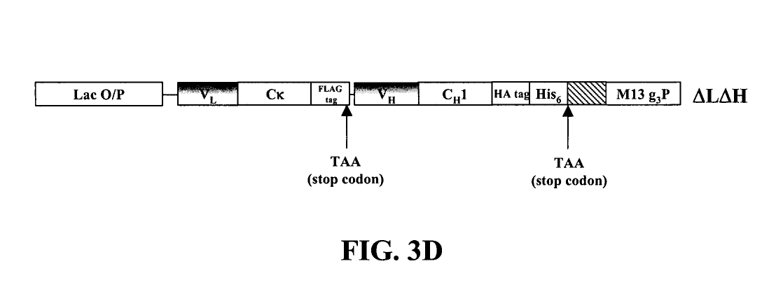

Expression and Preparation of the Different Fab Constructs: Expression of G5 and 12G3 Fabs in the context of the different leader sequences combinations described in FIG. 3A-D was carried out after infection of TG1 cells with the corresponding XL1-blue-produced phage constructs (see “Cloning of the Fab Fragments,” supra) essentially as described (Wu et al., ibid). More precisely, 300 ml of TG1 cells were used for each Fab construct. Supernatants were obtained after IPTG-induced cells were spun down at 3000 rpm for 30 min at 4° C. Periplasmic extracts were obtained as described (Wu et al., ibid) using 6.4 ml of resuspension buffer per construct. Cells pellets obtained at this step were then processed for the preparation of whole cell extracts as follows: pellets were resuspended in 6.4 ml of 30 mM Tris-HCl pH 8.0, 2 mM EDTA, 20% sucrose, 2 mg/ml lysozyme, 670U DNase I, submitted to 4 freeze/thaw cycles and spun down at 14000 rpm for 20 min at 4° C. Corresponding supernatants (“whole cell extracts”) were then recovered for analysis. The irrelevant antibody construct was processed in an identical fashion.

Determination of the [Fab+V_{L}-C_{L}] Concentration: In order to determine the concentration of both recombinant Fab (V_{H}/V_{L}) and V_{L}-C_{L} in the different samples (see “Expression and Preparation of the Different Fab Constructs,” supra), the following quantification ELISA was carried out: briefly, individual wells of a 96-well Biocoat Immunoplate (BD Bioscience, CA) were incubated with 2-fold serially diluted samples (supernatants, periplasmic extracts and whole cell extracts of G5 Fab, 12G3 Fab, irrelevant Fab and variants thereof) or standards (human IgG Fab (Cappel, Calif.) and V_{L}-C_{L} (see “Expression and Purification of a V_{L}-C_{L} Standard,” supra) at concentrations ranging from 50-0.39 ng/ml) for 1 hour at 37° C. Both standards were individually and systematically loaded on each assay plate. Incubation with a 1:1 mix of goat anti-human kappa horseradish peroxidase (Southern Biotech, AL; 1:5000 dilution) and of goat anti-human IgG horseradish peroxidase conjugate (Pierce, Ill., 1:12000 dilution) then followed. Horseradish peroxidase activity was detected with TMB substrate and the reaction quenched with 0.2 M H₂SO₄. Plates were read at 450 nm. In these conditions and for each assay plate, both V_{L}-C_{L} and human Fab standards exhibited essentially identical titration curves. This indicated that both Fab and V_{L}-C_{L} could be quantified together without any bias in the samples as long as the OD reading used for concentration calculations was in the overlapping regions of both standard curves. Results are indicated in Table 1.

Determination of the “Total” Fab Concentration: In order to determine the concentration of total Fab in the different samples (see “Expression and Preparation of the Different Fab Constructs,” supra), the following Fab-specific quantification ELISA was carried out: briefly, individual wells of a 96-well Maxisorp Immunoplate were coated with 150 ng of a sheep anti-human Fd (BioDesign, ME), blocked with 3% BSA/PBS for 2 h at 37° C. and incubated with 2-fold serially diluted samples (supernatants, periplasmic extracts and whole cell extracts of G5 Fab, 12G3 Fab, irrelevant Fab and variants thereof) or standards (human IgG Fab (Cappel, Calif.) at concentrations ranging from 12.5-0.098 ng/ml) for 1 hour at 37° C. Standards were systematically loaded on each assay plate. Incubation with a goat anti-human kappa horseradish peroxidase (Southern Biotech, AL; 1:5000 dilution) then followed. Horseradish peroxidase activity was detected with TMB substrate and the reaction quenched with 0.2 M H₂SO₄. Plates were read at 450 mm. Results are indicated in Table 1.

TABLE 1
Fab and V_{L}-C_{L} concentrations of 12G3, G5 and leader sequence variants
thereof in various E. Coli compartments.

| | Compartment Supernatant | | |
|---|---|---|---|
| Molecule | [Fab+ V_{L}-C_{L}]^{a} | [Total Fab]^{a} | [Active Fab]^{a} |
| Clone | | | |
| 12G3 “wild type” | 2.0 ± 0.4 | 0.021 ± 0.002 | 0.041 ± 0.003 |
| 12G3 ΔH | 1.6 ± 0.4 | <0.01 | <0.002 |
| 12G3 ΔL | 0.6 ± 0.1 | 0.013 ± 0.003 | 0.030 ± 0.001 |
| 12G3 ΔHΔL | 0.6 ± 0.1 | <0.01 | <0.002 |
| G5 “wild type” | 2.7 ± 0.2 | 0.033 ± 0.012 | 0.018 ± 0.002 |
| G5 ΔH | 2.7 ± 0.1 | 0.020 ± 0.008 | 0.011 ± 0.002 |
| G5 ΔL | 0.2 ± 0.005 | <0.01 | <0.01 |
| G5 ΔHΔL | 0.2 ± 0.009 | <0.01 | <0.01 |
| Irrelevant | 0.50 ± 0.03 | 0.053 ± 0.008 | N/A |
| | Compartment Periplasmic extract | | |
| Molecule | [Fab+ V_{L}-C_{L}]^{a} | [Total Fab]^{a} | [Active Fab]^{a} |
| Clone | | | |
| 12G3 “wild type” | 58.8 ± 1.0 | 1.04 ± 0.26 | 1.24 ± 0.52 |
| 12G3 ΔH | 57.7 ± 0.2 | 0.31 ± 0.01 | 0.03 ± 0.005 |
| 12G3 ΔL | 9.6 ± 0.2 | 0.22 ± 0.01 | 0.57 ± 0.15 |
| 12G3 ΔHΔL | 8.3 ± 0.2 | <0.01 | <0.002 |
| G5 “wild type” | 78.1 ± 4.0 | 1.71 ± 0.50 | 0.77 ± 0.15 |
| G5 ΔH | 45.4 ± 1.9 | 0.36 ± 0.03 | 0.16 ± 0.01 |
| G5 ΔL | 8.7 ± 0.2 | 0.13 ± 0.03 | 0.12 ± 0.01 |
| G5 ΔHΔL | 8.3 ± 0.05 | 0.013 ± 0.002 | 0.022 ± 0.004 |
| Irrelevant | 10.6 ± 0.3 | 0.87 ± 0.04 | N/A |
| | Compartment Whole cell extract | | |
| Molecule | [Fab+ V_{L}-C_{L}]^{a} | [Total Fab]^{a} | [Active Fab]^{a} |
| Clone | | | |
| 12G3 “wild type” | 91.2 ± 10.2 | 2.61 ± 0.61 | 2.36 ± 0.08 |
| 12G3 ΔH | 110.0 ± 14.2 | 2.03 ± 0.74 | 0.095 ± 0.008 |
| 12G3 ΔL | 32.9 ± 6.9 | 0.71 ± 0.02 | 1.73 ± 0.05 |
| 12G3 ΔHΔL | 25.9 ± 3.6 | 0.058 ± 0.032 | <0.002 |
| G5 “wild type” | 87.2 ± 7.4 | 3.66 ± 1.08 | 0.80 ± 0.06 |
| G5 ΔH | 94.8 ± 11.4 | 2.36 ± 0.89 | 0.41 ± 0.13 |
| G5 ΔL | 21.2 ± 1.4 | 0.38 ± 0.06 | 0.39 ± 0.05 |
| G5 ΔHΔL | 22.8 ± 3.7 | 0.19 ± 0.03 | 0.083 ± 0.009 |
| Irrelevant | 19.4 ± 1.6 | 1.54 ± 0.06 | N/A |

^{a}Concentrations represent the average of at least 2 individual measurements.

Determination of the “Active” Fab Concentration: In order to determine the concentration of “active” Fab (i.e., Fab that is able to recognize its cognate antigen) in the different G5 and 12G3 samples (see “Expression and Preparation of the Different Fab Constructs,” supra), the following quantification ELISA was carried out: briefly, individual wells of a 96-well Maxisorp Immunoplate were coated with 500 ng of human EphA2-Fc (Kinch et al., 2002, Metastasis 20:59-68), blocked with 3% BSA/PBS for 2 h at 37° C. and incubated with 2-fold serially diluted samples (supernatants, periplasmic extracts and whole cell extracts of G5 Fab, 12G3 Fab and variants thereof) or standards (G5 and 12G3 Fab standards (see “Production of G5 and 12G3 Fab Standards,” supra) at concentrations ranging from 100-1.56 ng/ml) for 1 hour at room temperature. Standards were individually and systematically loaded on each assay plate. Incubation with a goat anti-human kappa horseradish peroxidase (Southern Biotech, AL; 1:5000 dilution) then followed. Horseradish peroxidase activity was detected with TMB substrate and the reaction quenched with 0.2 M H₂SO₄. Plates were read at 450 nm. Results are indicated in Table I.

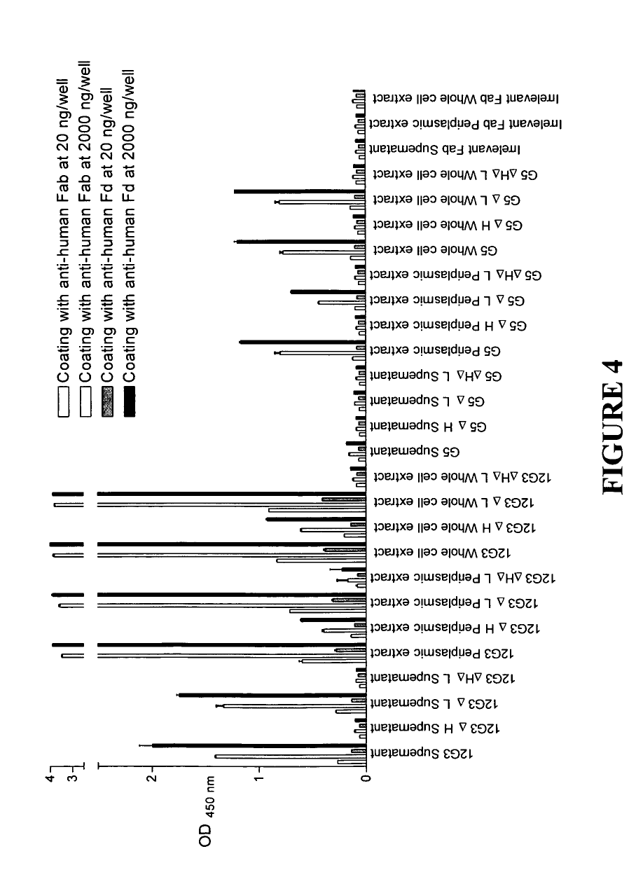

Capture ELISA: An EphA2-specific capture ELISA was carried out as follows: briefly, individual wells of a 96-well Maxisorp Immunoplate were coated with 20 or 2000 ng of a goat anti-human Fab antibody (Cappel, Calif.) or of a sheep anti-human Fd (BioDesign, ME), blocked with 3% BSA/PBS for 2 h at 37° C. and then incubated with 75 μl of the various samples (supernatants, periplasmic extracts and whole cell extracts of G5 Fab, 12G3 Fab, irrelevant Fab and variants thereof) for 2 hours at room temperature. 300 ng/well of biotinylated human EphA2-Fc was then added for 1.5 hours at room temperature. This was followed by incubation with a neutravidin-horseradish peroxidase (HRP) conjugate for 40 min at room temperature. HRP activity was detected with tetra methyl benzidine (TMB) substrate and the reaction quenched with 0.2 M H₂SO₄. Plates were read at 450 nm. Results are indicated in FIG. 4.

Results and Discussion

Standard methodology for the expression of secreted multi-protein complexes, for example an immunoglobulin (Ig), is to incorporate a host-cell appropriate signal sequence at the amino-terminal end of each protein of the complex and drive expression with one or more host-cell appropriate promoter. Using a standard single promoter-dicistronic gene arrangement, incorporating one signal sequence for each Ig chain, for the expression of Ig fragments in E. coli we observed that a major component of the Ig fragments produced was in fact “free” light chain. That is to say, the major component produced was light chain that was not found in association with a corresponding heavy chain (data not shown and Table 1). The presence of “free” light chain can be problematic as some or even most of the “free” light chain may be in the form of light chain dimers which can give spurious results in antigen binding studies. Thus, the presence of “free” light chain in Ig samples requires that samples be subjected to exhaustive purification procedures so that only properly assembled Ig fragments are assayed. An expression method which could reduce or even eliminate the production of “free” light chain would provide a significant advantage in the screening of large numbers of Ig clones which is often undertaken both during the initial screening for Ig molecules that bind a particular antigen and in subsequent optimization screens of a specific Ig molecule. Thus, a method for the production of sufficient Ig for screening purposes which does not incorporate the use of separate signal sequences for each Ig chain produced would be of benefit for the purpose of product development.

To investigate alternative methods of expressing Igs in a host cell a series of expression vector constructs were generated using only one or no signal sequences. Two human monoclonal antibodies (mAb12G3 and G5) raised against the human receptor tyrosine kinase EphA2 (Kinch et al., 2003) were used as model Ig molecules in this study. The amino acid sequences of the variable light (V_{L}) and variable heavy (V_{H}) genes of mAbs 12G3 and G5 are shown in FIG. 1. The Fab fragments of these two mAbs were cloned into a phage expression vector (FIG. 2). This vector allows the expression of Fab fragments that contain the first constant domain of a human γ1 heavy chain and the constant domain of a human kappa (κ) light chain in E. coli under the control of the lacZ promoter. For each Fab, four different constructs were generated that included (i) two separate g3 leader sequences in front of each the heavy and light chains (referred to as “wild type”, FIG. 3A), (ii) one g3 leader sequence in front of the heavy chain and none in front of the light chain (referred to as ΔL, FIG. 3B), (iii) one g3 leader sequence in front of the light chain and none in front of the heavy chain (referred to as ΔH, FIG. 3C) and (iv), no g3 leader sequences if front of both the light and heavy chains (referred to as ΔLΔH, FIG. 3D).

Expression of the Fab fragments from each of the different constructs described in FIG. 3 was carried out after electroporation of the phage DNA into E. coli. Assays were developed to distinguish between three Ig populations; a) Fab plus “free” light chain [Fab+V_{L}-V_{L}], which represents the sum total of all Ig produced, b) “total” Fab [Total Fab], which consists only of those Ig molecules that have formed a complete Fab fragment (i.e., a heavy chain paired with a light chain) and c) “active” Fab [Active Fab], which consists of only those Fab fragments capable of binding their epitope. The concentration of each of the above populations was determined in periplasmic and whole cell extracts, as well as, culture media supernatants.

Surprisingly, the main product produced when both leader sequences were present (“wild type” construct) was “free” light chain (compare [Fab+V_{L}-C_{L}] to [Total Fab] in Table 1), this despite the fact that a single promoter drives the expression of both Ig chains. Deletion of the leader sequence in front of the light chain (ΔL construct) resulted in a decrease of the “free” light chain produced (from 3 to 6 fold decrease for 12G3 and from 4 to 13 fold decrease for G5, compare [Fab+V_{L}-C_{L}] and [Total Fab] for wild type and ΔL in Table 1). Although there was also a decrease in the concentration of “active” Fab, the decreases were much smaller (a 1.4 to 2.2 reduction for 12G3 and a 1.8 to 6.4 fold reduction for G5). Note that for both the wild type and the ΔL construct, most of the “total” Fab produced was generally found to be active (compare [Total Fab] to [Active Fab] in Table 1). This mirrors the wild type situation for 12G3 whereas in the case of G5, the [active Fab]: [total Fab] ratio is significantly higher than in the wild type situation. Thus, deletion of the light chain leader sequence seems to favor “active” Fab production over “free” light chain production (the major product in the two leader sequence “wild type” construct).

In contrast, when the leader sequence in front of the heavy chain (ΔH construct) was deleted, the concentration of “free” light chain remained largely unaffected with only a modest decrease in the periplasmic extract of G5 and a slight increase in the whole cell extracts of both G5 and 12G3 seen in comparison to the “wild type” construct (compare [Fab+V_{L}-C_{L}] and [Total Fab] for wild type and ΔH in Table 1). Additionally, removal of the heavy chain leader sequence generally resulted in a large (1.5 to 5 fold for G5) to dramatic (20 to 41 fold for 12G3) reduction in the “active” Fab concentration when compared to the “wild type” construct (compare [Total Fab] to [Active Fab] in Table 1). Furthermore, in the absence of a heavy chain leader sequence the “active” Fab concentration is significantly less than the corresponding “total” Fab concentration (10 to 20 fold less for 12G3 and 2 to 5.5 fold less for G5). This mirrors the wild type situation for G5 where the [Active Fab][Total Fab] ratio is quite low, indicating that a large portion of the Fab fragments are non-functional. However, in the case of 12G3, the [Active Fab][Total Fab] ratio is significantly lower than seen with G5, indicating that an even larger portion of the Fab fragments are non-functional.

An antigen-specific capture ELISA assay was utilized to examine the relative amount of functional Fab fragments in each sample. It can be seen that the signal obtained from the ΔL construct for both G5 and 12G3 is very nearly identical to that obtained from the “wild type” construct, this in spite of the lower “active” Fab concentrations seen for the ΔL construct samples (FIG. 4). Interestingly, for G5 the removal of the heavy chain leader sequence (ΔH construct) results in roughly the same decrease in “active” Fab concentration as the removal of the light chain leader sequence (ΔL construct). However, only the ΔL construct results in a good signal in the antigen-specific capture ELISA. In the case of 12G3 Fab expression, the ΔH construct exhibits a much weaker signal in the antigen-specific capture ELISA than both the “wild type” and ΔL constructs. This may be due partly to the much more profound decrease in the “active” Fab concentration upon deletion of the heavy chain leader sequence.

The deletion of both leader sequences results in a dramatic decrease in the production of both total Fab and “active” Fab. This result is not unexpected in light of prior studies on the expression of numerous secreted proteins including Igs.

The importance of decreasing the concentration of “free” light chain is exemplified by the results of the antigen-specific capture ELISA (FIG. 4) where it can be seen that removal of the light chain leader sequence had virtually no effect on the signal obtained even though this results in a significant decrease in “active” Fab concentration. Since it is clear from Table 1 that removal of the light chain leader sequence profoundly reduced the amount of “free” light chain produced, it can be inferred that the use of a single signal sequence on the heavy chain of an Ig can result in the production of a significantly more homogenous population of active and properly assembled Ig. This greatly facilitates direct screening and reduces the interfering effect of free subunits.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above may be used in various combinations. All publications, patents, patent applications, or other documents cited in this application are incorporated by reference in their entirety for all purposes to the same extent as if each individual publication, patent, patent application, or other document were individually indicated to be incorporated by reference for all purposes.

1. An isolated or recombinant polynucleotide molecule comprising a promoter region operably linked to a transcription unit, said transcription unit comprising:
a. a first DNA sequence encoding a first polypeptide, and
b. a second DNA sequence encoding a second polypeptide,
wherein either the first or the second DNA sequence but not both, additionally encode a secretion signal sequence operably linked to the DNA sequence encoding said first or second polypeptide.

2. The polynucleotide molecule of claim 1, wherein said first DNA sequence encodes a immunoglobulin light chain or a fragment thereof, and said second DNA sequence encodes a immunoglobulin heavy chain or a fragment thereof.

3. The polynucleotide molecule of claim 1, wherein said first polypeptide is an immunoglobulin heavy chain or a fragment thereof and said second polypeptide is an immunoglobulin light chain or a fragment thereof.

4. The polynucleotide molecule of claim 1, 2 or 3, wherein said first DNA sequence further incorporates at least one polynucleotide encoding a non-immunoglobulin molecule.

5. The polynucleotide molecule of claim 1, 2 or 3, wherein said second DNA sequence further incorporates at least one polynucleotide encoding a non-immunoglobulin molecule.

6. The polynucleotide molecule of claim 1, 2 or 3, wherein said first and second DNA sequence are dicistronic.

7. The polynucleotide molecule of claim 1, 2 or 3, further comprising a second promoter region operable linked to said second DNA sequence.

8. The polynucleotide molecule of claim 2 or 3, wherein said immunoglobulin light and heavy chains or fragments thereof are selected from the group consisting of: a) rodent immunoglobulins; b) primate immunoglobulins; c) chimeric immunoglobulins; d) humanized immunoglobulins; and e) human immunoglobulins.

9. A recombinant expression vector comprising the polynucleotide molecule of claim 1.

10. A recombinant expression vector comprising the polynucleotide molecule of claim 2 or 3.

11. A method of producing a multimeric protein comprising culturing a host cell that has been transformed or transfected with the recombinant expression vector of claim 9, under culture conditions such that said host cell produces said multimeric protein.

12. The method of claim 11, wherein said host cell is a prokaryote cell.

13. The method of claim 12, wherein said prokaryote cell is an E. coli cell.

14. A method of producing an antibody comprising culturing a host cell that has been transformed or transfected with the recombinant expression vector of claim 10, under culture conditions such that said host cell produces said antibody.

15. The method of claim 14, wherein said host cell is a prokaryote cell.

16. The method of claim 15, wherein said prokaryote cell is an E. coli cell.

17. The method of claim 14, wherein the produced antibody is selected from the group consisting of: a) full length antibody; b) Fd fragment; c) Fv fragment; d) Fab fragment; and (e) F(ab)₂.

18. The method of claim 17, wherein the produced antibody is selected from the group consisting of: a) rodent antibodies; b) primate antibodies; c) a chimeric antibodies; d) humanized antibodies and e) human antibodies.

19. The method of claim 14, further comprising the step of recovering the produced antibody.

20. The method of claim 19, wherein said antibody is recovered from at least one location selected from the group consisting of: the periplasm, the whole cell and the culture media.

21. A method of reducing the production of immunoglobulin light chain not associated with heavy chain during the production of an antibody comprising culturing a host cell that has been transformed with the recombinant expression vector of claim 10 under culture conditions such that said host cell produces said antibody or fragment thereof, wherein said expression vector encodes a immunoglobulin light chain that is not operably linked to a secretion signal sequence.

22. The method of claim 21, wherein said host cell is a prokaryote cell.

23. The method of claim 22, wherein said prokaryote cell is an E. coli cell.

24. The method of claim 21, wherein the immunoglobulin light chain reduced by the method is a full length light chain or a functional fragment thereof.

25. A method of reducing an accumulation of immunoglobulin heavy chain during the production of an antibody comprising culturing a host cell that has been transformed with the recombinant expression vector of claim 10 under culture conditions such that said host cell produces said antibody, wherein said expression vector encodes a immunoglobulin heavy chain that is not operably linked to a secretion signal sequence.

26. The method of claim 25, wherein said host cell is a prokaryote cell.

27. The method of claim 26, wherein said prokaryote cell is an E. coli cell.

28. The method of claim 25, wherein the immunoglobulin heavy chain reduced by the method is a full length heavy chain or a functional fragment thereof.

29. A method of increasing the ratio of active antibody to total immunoglobulin chains during the production of an antibody comprising culturing a host cell that has been transformed with the recombinant expression vector of claim 10 under culture conditions such that said host cell produces said antibody, wherein said expression vector encodes a immunoglobulin light chain that is not operably linked to a secretion signal sequence.

30. The method of claim 29, wherein said host cell is a prokaryote cell.

31. The method of claim 30, wherein said prokaryote cell is an E. coli cell.
